(19) United States
(12) Patent Application Publication
(10) Pub. No.: US 2013/0065970 A1
(43) Pub. Date: Mar. 14, 2013

(54) COMPOSITIONS AND PRODUCTS CONTAINING ESTOLIDE COMPOUNDS

(71) Applicants: Jakob BREDSGUARD, Irvine, CA (US); Travis THOMPSON, Anaheim, CA (US); Kelly PARSON, Irvine, CA (US)

(72) Inventors: Jakob BREDSGUARD, Irvine, CA (US); Travis THOMPSON, Anaheim, CA (US); Kelly PARSON, Irvine, CA (US)

(21) Appl. No.: 13/534,424

(22) Filed: Jun. 27, 2012

Related U.S. Application Data
(60) Provisional application No. 61/505,913, filed on Jul. 8, 2011.

(51) Int. Cl. A61K 8/37; A61Q 19/10; A61Q 1/00; A61Q 9/00; A61Q 5/00; A61Q 11/00; A61Q 19/00; A61Q 15/00

(52) U.S. Cl. 514/785

(57) ABSTRACT

Estolide compounds that may be suitable for use in personal care and cosmetic formulations, and method of preparing the same. The estolides can be tailored to exhibit the desired viscometric and lubricity properties, while retaining or even improving other properties desirable in such products.

### CROSS REFERENCE TO RELATED APPLICATIONS

This application claims the benefit under 35 U.S.C. §119(e) of U.S. Provisional Patent Application No. 61/505,913, filed Jul. 8, 2011, which is incorporated herein by reference in its entirety for all purposes.

### FIELD

The present disclosure relates to estolide compounds, compositions, and methods of making the same. The estolides described herein may be suitable for use in personal care products.

### BACKGROUND

Personal care products generally include compositions used for skin care and maintenance, cleansing, odor improvement, hair removal, hair care and maintenance, care and maintenance of mucous membranes, and decorative cosmetics. Most personal care products on the market contain many types of compounds that vary by structure, chemistry, and raw material source (synthetic or natural) that are combined to provide products with many different desired functions.

### SUMMARY

Described herein are personal care products comprising one or more estolide compounds, and methods of making and using the same.

In certain embodiments, the composition comprises at least one estolide compound of Formula I:

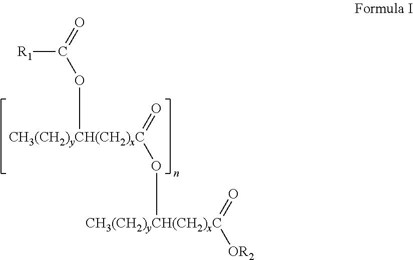

wherein

x is, independently for each occurrence, an integer selected from 0, 1, 2, 3, 4, 5, 6, 7, 8, 9, 10, 11, 12, 13, 14, 15, 16, 17, 18, 19, and 20;

y is, independently for each occurrence, an integer selected from 0, 1, 2, 3, 4, 5, 6, 7, 8, 9, 10, 11, 12, 13, 14, 15, 16, 17, 18, 19, and 20;

n is an integer selected from 0, 1, 2, 3, 4, 5, 6, 7, 8, 9, 10, 11, and 12;

R₁ is an optionally substituted alkyl that is saturated or unsaturated, and branched or unbranched; and

R₂ is selected from hydrogen and optionally substituted alkyl that is saturated or unsaturated, and branched or unbranched;

wherein each fatty acid chain residue of said at least one compound is independently optionally substituted.

In certain embodiments, the composition comprises at least one estolide compound of

Formula II:

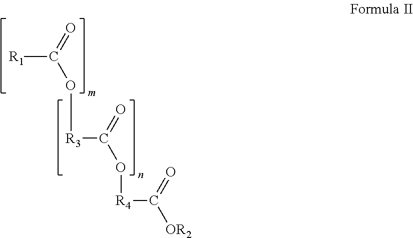

wherein

m is an integer equal to or greater than 1;

n is an integer equal to or greater than 0;

R₁, independently for each occurrence, is an optionally substituted alkyl that is saturated or unsaturated, and branched or unbranched;

R₂ is selected from hydrogen and optionally substituted alkyl that is saturated or unsaturated, and branched or unbranched; and

R₃ and R₄, independently for each occurrence, are selected from optionally substituted alkyl that is saturated or unsaturated, and branched or unbranched.

In certain embodiments, the composition comprises at least one estolide compound of Formula III:

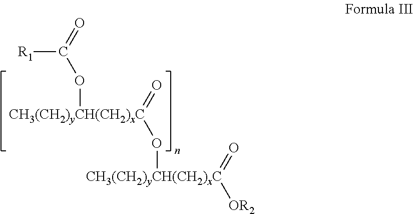

wherein

x is, independently for each occurrence, an integer selected from 0, 1, 2, 3, 4, 5, 6, 7, 8, 9, 10, 11, 12, 13, 14, 15, 16, 17, 18, 19, and 20;

y is, independently for each occurrence, an integer selected from 0, 1, 2, 3, 4, 5, 6, 7, 8, 9, 10, 11, 12, 13, 14, 15, 16, 17, 18, 19, and 20;

n is an integer equal to or greater than 0;

R₁ is an optionally substituted alkyl that is saturated or unsaturated, and branched or unbranched; and

R₂ is selected from hydrogen and optionally substituted alkyl that is saturated or unsaturated, and branched or unbranched;

wherein each fatty acid chain residue of said at least one compound is independently optionally substituted.

### DETAILED DESCRIPTION

With respect to cosmetics and personal care products, one class of additive compound is targeted at altering or modifying the rheological properties of the product that add to consumer appeal. For example, additives that provide sufficient viscosity may be needed, especially for those formulations where the viscosity without additives is close to that of the pure solvent (e.g., water). Because merely altering viscosity may not be sufficient, modifiers are selected to provide certain desired rheological properties for the formulation that depend on its nature, the mode of delivery, type of flow, and the aesthetic appeal of final application. In certain instances, low molecular weight surfactants may be used to modify rheological properties. However, such additives may have to be used in large concentrations, which can result in relatively high costs and an adverse impact on the environment (e.g., water pollution). The properties of the estolide compounds and compositions described herein may make them suitable for use in certain personal care applications.

It may be desirable for compounds used in cosmetic and body care preparations to meet several desirable qualities. First, they may exhibit high compatibility and, if possible, biodegradability. In some cases, however, compounds tested in the industry fail to meet these standards. Second, it may be desirable for cosmetic compounds to be universally useable in aqueous, emulsoidal, alcoholic and oil-containing bases. Suitable cosmetic compounds may also be readily proces sable and provide a final rheology that allows the product to be easily applied and removed. Finally, such desirable compounds may further demonstrate stable rheology and an unchanging physical and chemical quality when exposed to long-term storage and changes in pH and temperature. In certain embodiments, the estolide compounds and compositions described herein meet one or more of these desired characteristics.

In certain embodiments, the estolide compounds and compositions described herein are partially or fully biodegradable and thereby pose diminished risk to the environment. In certain embodiments, the compositions meet guidelines set for by the Organization for Economic Cooperation and Development (OECD) for degradation and accumulation testing. The OECD has indicated that several tests may be used to determine the “ready biodegradability” of organic chemicals. Aerobic ready biodegradability by OECD 301D measures the mineralization of the test sample to CO₂ in closed aerobic microcosms that simulate an aerobic aquatic environment, with microorganisms seeded from a waste-water treatment plant. OECD 301D is considered representative of most aerobic environments that are likely to receive waste materials. Aerobic “ultimate biodegradability” can be determined by OECD 302D. Under OECD 302D, microorganisms are pre-acclimated to biodegradation of the test material during a pre-incubation period, then incubated in sealed vessels with relatively high concentrations of microorganisms and enriched mineral salts medium. OECD 302D ultimately determines whether the test materials are completely biodegradable, albeit under less stringent conditions than “ready biodegradability” assays.

As used in the present specification, the following words, phrases and symbols are generally intended to have the meanings as set forth below, except to the extent that the context in which they are used indicates otherwise. The following abbreviations and terms have the indicated meanings throughout:

A dash (“—”) that is not between two letters or symbols is used to indicate a point of attachment for a substituent. For example, —C(O)NH₂ is attached through the carbon atom.

“Alkoxy” by itself or as part of another substituent refers to a radical —OR³¹ where R³¹ is alkyl, cycloalkyl, cycloalkylalkyl, aryl, or arylalkyl, which can be substituted, as defined herein. In some embodiments, alkoxy groups have from 1 to 8 carbon atoms. In some embodiments, alkoxy groups have 1, 2, 3, 4, 5, 6, 7, or 8 carbon atoms. Examples of alkoxy groups include, but are not limited to, methoxy, ethoxy, propoxy, butoxy, cyclohexyloxy, and the like.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2- yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

Unless otherwise indicated, the term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 40 carbon atoms, in certain embodiments, from 1 to 22 or 1 to 18 carbon atoms, in certain embodiments, from 1 to 16 or 1 to 8 carbon atoms, and in certain embodiments from 1 to 6 or 1 to 3 carbon atoms. In certain embodiments, an alkyl group comprises from 8 to 22 carbon atoms, in certain embodiments, from 8 to 18 or 8 to 16. In some embodiments, the alkyl group comprises from 3 to 20 or 7 to 17 carbons. In some embodiments, the alkyl group comprises 1, 2, 3, 4, 5, 6, 7, 8, 9, 10, 11, 12, 13, 14, 15, 16, 17, 18, 19, 20, 21, or 22 carbon atoms.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered non-aromatic heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 5 to 20 carbon atoms, and in certain embodiments, from 5 to 12 carbon atoms. In certain embodiments, an aryl group can comprise 5, 6, 7, 8, 9, 10, 11, 12, 13, 14, 15, 16, 17, 18, 19, or 20 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

Estolide “base oil” and “base stock”, unless otherwise indicated, refer to any composition comprising one or more estolide compounds. It should be understood that an estolide “base oil” or “base stock” is not limited to compositions for a particular use, and may generally refer to compositions comprising one or more estolides, including mixtures of estolides. Estolide base oils and base stocks can also include compounds other than estolides.

“Compounds” refers to compounds encompassed by structural Formula I, II, and III herein and includes any specific compounds within the formula whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

For the purposes of the present disclosure, “chiral compounds” are compounds having at least one center of chirality (i.e. at least one asymmetric atom, in particular at least one asymmetric C atom), having an axis of chirality, a plane of chirality or a screw structure. “Achiral compounds” are compounds which are not chiral.

Compounds of Formula I, II, and III include, but are not limited to, optical isomers of compounds of Formula I, II, and III, racemates thereof, and other mixtures thereof. In such embodiments, the single enantiomers or diastereomers, i.e., optically active forms, can be obtained by asymmetric synthesis or by resolution of the racemates. Resolution of the racemates may be accomplished by, for example, chromatography, using, for example a chiral high-pressure liquid chromatography (HPLC) column. However, unless otherwise stated, it should be assumed that Formula I, II, and III cover all asymmetric variants of the compounds described herein, including isomers, racemates, enantiomers, diastereomers, and other mixtures thereof. In addition, compounds of Formula I, II and III include Z- and E-forms (e.g., cis- and trans-forms) of compounds with double bonds. The compounds of Formula I, II, and III may also exist in several tautomeric forms including the enol form, the keto form, and mixtures thereof. Accordingly, the chemical structures depicted herein encompass all possible tautomeric forms of the illustrated compounds.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl. In certain embodiments, a cycloalkyl group is a C₅, C₆, C₇, C₈, C₉, C₁₀, C₁₁, C₁₂, C₁₃, C₁₄, or C₁₅ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Halogen” refers to a fluoro, chloro, bromo, or iodo group.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, f3-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments, a heteroaryl group is a 5-, 6-, 7-, 8-, 9-, 10-, 11-, 12-, 13-, 14-, 15-, 16-, 17-, 18-, 19-, or 20-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Heterocycloalkylalkyl” by itself or as part of another substituent refers to an acyclic alkyl radical in which one of the hydrogen atoms bonded to a carbon atom, typically a terminal or sp³ carbon atom, is replaced with a heterocycloalkyl group. Where specific alkyl moieties are intended, the nomenclature heterocycloalkylalkanyl, heterocycloalkylalkenyl, or heterocycloalkylalkynyl is used. In certain embodiments, a heterocycloalkylalkyl group is a 6- to 30-membered heterocycloalkylalkyl, e.g., the alkanyl, alkenyl, or alkynyl moiety of the heterocycloalkylalkyl is 1- to 10-membered and the heterocycloalkyl moiety is a 5- to 20-membered heterocycloalkyl, and in certain embodiments, 6- to 20-membered heterocycloalkylalkyl, e.g., the alkanyl, alkenyl, or alkynyl moiety of the heterocycloalkylalkyl is 1- to 8-membered and the heterocycloalkyl moiety is a 5- to 12-membered heterocycloalkyl.

“Mixture” refers to a collection of molecules or chemical substances. Each component in a mixture can be independently varied. A mixture may contain, or consist essentially of, two or more substances intermingled with or without a constant percentage composition, wherein each component may or may not retain its essential original properties, and where molecular phase mixing may or may not occur. In mixtures, the components making up the mixture may or may not remain distinguishable from each other by virtue of their chemical structure.

“Parent aromatic ring system” refers to an unsaturated cyclic or polycyclic ring system having a conjugated it (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Substituted” refers to a group in which one or more hydrogen atoms are independently replaced with the same or different substituent(s). Examples of substituents include, but are not limited to, —R⁶⁴, —R⁶⁰, —O⁻, —OH, ═O, —OR⁶⁰, —SR⁶⁰, —S⁻, ═S, —NR⁶⁰R⁶¹, ═NR⁶⁰, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S (O)₂R⁶⁰, —NR⁶³C(O)R⁶⁰, and —S(O)R⁶⁰;

wherein each —R⁶⁴ is independently a halogen; each R⁶⁰ and R⁶¹ are independently alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, or substituted heteroarylalkyl, or R⁶⁰ and R⁶¹ together with the nitrogen atom to which they are bonded form a heterocycloalkyl, substituted heterocycloalkyl, heteroaryl, or substituted heteroaryl ring, and R⁶² and R⁶³ are independently alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, or substituted heteroarylalkyl, or R⁶² and R⁶³ together with the atom to which they are bonded form one or more heterocycloalkyl, substituted heterocycloalkyl, heteroaryl, or substituted heteroaryl rings;

wherein the “substituted” substituents, as defined above for R⁶⁰, R⁶¹, R⁶², and R⁶³, are substituted with one or more, such as one, two, or three, groups independently selected from alkyl, -alkyl-OH, —O-haloalkyl, -alkyl-NH₂, alkoxy, cycloalkyl, cycloalkylalkyl, heterocycloalkyl, heterocycloalkylalkyl, aryl, heteroaryl, arylalkyl, heteroarylalkyl, —O⁻, —OH, ═O, —O-alkyl, —O-aryl, —O-heteroarylalkyl, —O-cycloalkyl, —O-heterocycloalkyl, —SH, —S⁻, ═S, —S-alkyl, —S-aryl, —S-heteroarylalkyl, —S-cycloalkyl, —S-heterocycloalkyl, —NH₂, ═NH, —CN, —CF₃, —OCN, —SCN, —NO, —NO₂, ═N₂, —N₃, —S(O)₂O, —S(O)₂, —S(O)₂OH, —OS(O₂)O⁻, —SO₂(alkyl), —SO₂(phenyl), —SO₂(haloalkyl), —SO₂NH₂, —SO₂NH(alkyl), —SO₂NH(phenyl), —P(O)(O⁻)₂, —P(O)(O-alkyl)(O⁻), —OP(O)(O-alkyl)(O-alkyl), —CO₂H, —C(O)O(alkyl), —CON(alkyl)(alkyl), —CONH(alkyl), —CONH₂, —C(O)(alkyl), —C(O)(phenyl), —C(O)(haloalkyl), —OC(O)(alkyl), —N(alkyl)(alkyl), —NH(alkyl), —N(alkyl)(alkylphenyl), —NH(alkylphenyl), —NHC(O)(alkyl), —NHC(O)(phenyl), —N(alkyl)C(O)(alkyl), and —N(alkyl)C(O)(phenyl).

The terms “cosmetic,” “cosmetic composition,” and “cosmetic formulation,” unless otherwise stated, shall mean any substance or preparation intended to be placed in contact with the various external parts of the human body, including the epidermis, hair system, nails, and lips. “Cosmetics” may be placed with the intended purpose of cleaning, perfuming, beautifying, changing appearance and/or correcting odors and/or protecting or keeping the contacted portions of the human body in good condition.

The term “personal care product” shall reference any cosmetic and/or toiletry product that may be used on or in contact with the hair, skin, nails, teeth, or oral cavity, and includes effective concentrations of one or more of the compositions described herein. Personal care products may include, for example, cosmetics, floating bath oils, after shaves, creams, lotions, deodorants, including stick deodorants, pre-electric shave lotions, after-shave lotions, antiperspirants, shampoos, hair-coloring products, conditioners, rinses and related products, among others, including skin care products, eye makeups, body shampoos, protective skin formulations, lipsticks, lip glosses, after-bath splashes, presun and sun products, including sunscreens.

As used in this specification and the appended claims, the articles “a,” “an,” and “the” include plural referents unless expressly and unequivocally limited to one referent.

All numerical ranges herein include all numerical values and ranges of all numerical values within the recited range of numerical values.

The present disclosure relates to personal care products comprising at least one estolide compound, compositions comprising at least one estolide compound, and methods of making the same. In certain embodiments the at least one estolide compound is selected from compounds of Formula I:

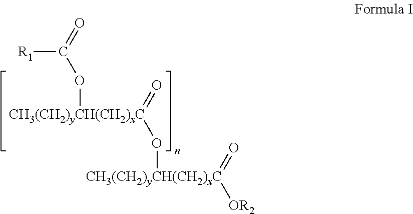

wherein

x is, independently for each occurrence, an integer selected from 0, 1, 2, 3, 4, 5, 6, 7, 8, 9, 10, 11, 12, 13, 14, 15, 16, 17, 18, 19, and 20;

y is, independently for each occurrence, an integer selected from 0, 1, 2, 3, 4, 5, 6, 7, 8, 9, 10, 11, 12, 13, 14, 15, 16, 17, 18, 19, and 20;

n is an integer selected from 0, 1, 2, 3, 4, 5, 6, 7, 8, 9, 10, 11, and 12;

R₁ is an optionally substituted alkyl that is saturated or unsaturated, and branched or unbranched; and

R₂ is selected from hydrogen and optionally substituted alkyl that is saturated or unsaturated, and branched or unbranched;

wherein each fatty acid chain residue of said at least one compound is independently optionally substituted.

In certain embodiments the at least one estolide compound is selected from compounds of Formula II:

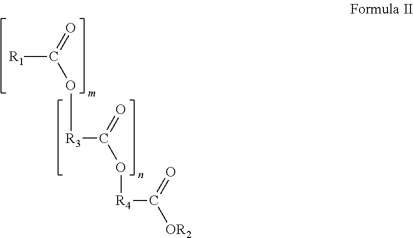

wherein

m is an integer greater than or equal to 1;

n is an integer greater than or equal to 0;

R₁, independently for each occurrence, is an optionally substituted alkyl that is saturated or unsaturated, and branched or unbranched;

R₂ is selected from hydrogen and optionally substituted alkyl that is saturated or unsaturated, and branched or unbranched; and

R₃ and R₄, independently for each occurrence, are selected from optionally substituted alkyl that is saturated or unsaturated, and branched or unbranched.

In certain embodiments the at least one estolide compound is selected from compounds of Formula III:

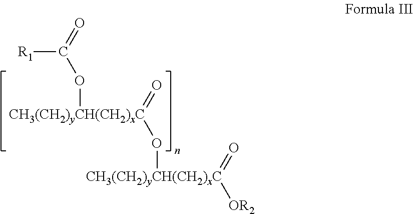

wherein

x is, independently for each occurrence, an integer selected from 0 to 20;

y is, independently for each occurrence, an integer selected from 0 to 20;

n is an integer greater than or equal to 0;

R₁ is an optionally substituted alkyl that is saturated or unsaturated, and branched or unbranched; and

R₂ is selected from hydrogen and optionally substituted alkyl that is saturated or unsaturated, and branched or unbranched;

wherein each fatty acid chain residue of said at least one compound is independently optionally substituted.

In certain embodiments, the composition comprises at least one estolide compound of Formula I, II, or III where R₁ is hydrogen.

The terms “chain” or “fatty acid chain” or “fatty acid chain residue,” as used with respect to the estolide compounds of Formula I, II, and III, refer to one or more of the fatty acid residues incorporated in estolide compounds, e.g., R₃ or R₄ of Formula II, or the structures represented by CH₃(CH₂)_{y}CH(CH₂)ₓC(O)O— in Formula I and III.

The R₁ in Formula I, II, and III at the top of each Formula shown is an example of what may be referred to as a “cap” or “capping material,” as it “caps” the top of the estolide. Similarly, the capping group may be an organic acid residue of general formula —OC(O)-alkyl, i.e., a carboxylic acid with a substituted or unsubstituted, saturated or unsaturated, and/or branched or unbranched alkyl as defined herein, or a formic acid residue. In certain embodiments, the “cap” or “capping group” is a fatty acid. In certain embodiments, the capping group, regardless of size, is substituted or unsubstituted, saturated or unsaturated, and/or branched or unbranched. The cap or capping material may also be referred to as the primary or alpha (a) chain.

Depending on the manner in which the estolide is synthesized, the cap or capping group alkyl may be the only alkyl from an organic acid residue in the resulting estolide that is unsaturated. In certain embodiments, it may be desirable to use a saturated organic or fatty-acid cap to increase the overall saturation of the estolide and/or to increase the resulting estolide's stability. For example, in certain embodiments, it may be desirable to provide a method of providing a saturated capped estolide by hydrogenating an unsaturated cap using any suitable methods available to those of ordinary skill in the art. Hydrogenation may be used with various sources of the fatty-acid feedstock, which may include mono- and/or polyunsaturated fatty acids. Without being bound to any particular theory, in certain embodiments, hydrogenating the estolide may help to improve the overall stability of the molecule. However, a fully-hydrogenated estolide, such as an estolide with a larger fatty acid cap, may exhibit increased melting point temperatures. In certain embodiments, it may be desirable to offset any loss in desirable melting-point characteristics by using shorter, saturated capping materials.

The R₄C(O)O— of Formula II or structure CH₃(CH₂)_{y}CH(CH₂)ₓC(O)O— of Formula I and III serve as the “base” or “base chain residue” of the estolide. Depending on the manner in which the estolide is synthesized, the base organic acid or fatty acid residue may be the only residue that remains in its free-acid form after the initial synthesis of the estolide. However, in certain embodiments, in an effort to alter or improve the properties of the estolide, the free acid may be reacted with any number of substituents. For example, it may be desirable to react the free acid estolide with alcohols, glycols, amines, or other suitable reactants to provide the corresponding ester, amide, or other reaction products. The base or base chain residue may also be referred to as tertiary or gamma (y) chains.

The R₃C(O)O— of Formula II or structure CH₃(CH₂)_{y}CH(CH₂)ₓC(O)O— of Formula I and III are linking residues that link the capping material and the base fatty-acid residue together. There may be any number of linking residues in the estolide, including when n=0 and the estolide is in its dimer form. Depending on the manner in which the estolide is prepared, a linking residue may be a fatty acid and may initially be in an unsaturated form during synthesis. In some embodiments, the estolide will be formed when a catalyst is used to produce a carbocation at the fatty acid's site of unsaturation, which is followed by nucleophilic attack on the carbocation by the carboxylic group of another fatty acid. In some embodiments, it may be desirable to have a linking fatty acid that is monounsaturated so that when the fatty acids link together, all of the sites of unsaturation are eliminated. The linking residue(s) may also be referred to as secondary or beta (β) chains.

In certain embodiments, the cap is an acetyl group, the linking residue(s) is one or more fatty acid residues, and the base chain residue is a fatty acid residue. In certain embodiments, the linking residues present in an estolide differ from one another. In certain embodiments, one or more of the linking residues differs from the base chain residue.

As noted above, in certain embodiments, suitable unsaturated fatty acids for preparing the estolides may include any mono- or polyunsaturated fatty acid. For example, monounsaturated fatty acids, along with a suitable catalyst, will form a single carbocation that allows for the addition of a second fatty acid, whereby a single link between two fatty acids is formed. Suitable monounsaturated fatty acids may include, but are not limited to, palmitoleic acid (16:1), vaccenic acid (18:1), oleic acid (18:1), eicosenoic acid (20:1), erucic acid (22:1), and nervonic acid (24:1). In addition, in certain embodiments, polyunsaturated fatty acids may be used to create estolides. Suitable polyunsaturated fatty acids may include, but are not limited to, hexadecatrienoic acid (16:3), alpha-linolenic acid (18:3), stearidonic acid (18:4), eicosatrienoic acid (20:3), eicosatetraenoic acid (20:4), eicosapentaenoic acid (20:5), heneicosapentaenoic acid (21:5), docosapentaenoic acid (22:5), docosahexaenoic acid (22:6), tetracosapentaenoic acid (24:5), tetracosahexaenoic acid (24:6), linoleic acid (18:2), gamma-linoleic acid (18:3), eicosadienoic acid (20:2), dihomo-gamma-linolenic acid (20:3), arachidonic acid (20:4), docosadienoic acid (20:2), adrenic acid (22:4), docosapentaenoic acid (22:5), tetracosatetraenoic acid (22:4), tetracosapentaenoic acid (24:5), pinolenic acid (18:3), podocarpic acid (20:3), rumenic acid (18:2), alpha-calendic acid (18:3), beta-calendic acid (18:3), jacaric acid (18:3), alpha-eleostearic acid (18:3), beta-eleostearic (18:3), catalpic acid (18:3), punicic acid (18:3), rumelenic acid (18:3), alpha-parinaric acid (18:4), beta-parinaric acid (18:4), and bosseopentaenoic acid (20:5). In certain embodiments, hydroxy fatty acids may be polymerized or homopolymerized by reacting the carboxylic acid functionality of one fatty acid with the hydroxy functionality of a second fatty acid. Exemplary hydroxyl fatty acids include, but are not limited to, ricinoleic acid, 6-hydroxystearic acid, 9,10-dihydroxystearic acid, 12-hydroxystearic acid, and 14-hydroxystearic acid.

The process for preparing the estolide compounds described herein may include the use of any natural or synthetic fatty acid source. However, it may be desirable to source the fatty acids from a renewable biological feedstock. For example, suitable starting materials of biological origin include, but are not limited to, plant fats, plant oils, plant waxes, animal fats, animal oils, animal waxes, fish fats, fish oils, fish waxes, algal oils and mixtures of two or more thereof. Other potential fatty acid sources include, but are not limited to, waste and recycled food-grade fats and oils, fats, oils, and waxes obtained by genetic engineering, fossil fuel-based materials and other sources of the materials desired.

In certain embodiments, the estolide compounds described herein may be prepared from non-naturally occurring fatty acids derived from naturally occurring feedstocks. In certain embodiments, the estolides are prepared from synthetic fatty acid reactants derived from naturally occurring feedstocks such as vegetable oils. For example, the synthetic fatty acid reactants may be prepared by cleaving fragments from larger fatty acid residues occurring in natural oils such as triglycerides using, for example, a cross-metathesis catalyst and alpha-olefin(s). The resulting truncated fatty acid residue(s) may be liberated from the glycerine backbone using any suitable hydrolytic and/or transesterification processes known to those of skill in the art. An exemplary fatty acid reactant includes 9-dodecenoic acid, which may be prepared via the cross metathesis of an oleic acid residue with 1-butene.

In some embodiments, the compound comprises chain residues of varying lengths. In some embodiments, x is, independently for each occurrence, an integer selected from 0 to 20, 0 to 18, 0 to 16, 0 to 14, 1 to 12, 1 to 10, 2 to 8, 6 to 8, or 4 to 6. In some embodiments, x is, independently for each occurrence, an integer selected from 7 and 8. In some embodiments, x is, independently for each occurrence, an integer selected from 0, 1, 2, 3, 4, 5, 6, 7, 8, 9, 10, 11, 12, 13, 14, 15, 16, 17, 18, 19, and 20. In certain embodiments, for at least one chain residue, x is an integer selected from 7 and 8.

In some embodiments, y is, independently for each occurrence, an integer selected from 0 to 20, 0 to 18, 0 to 16, 0 to 14, 1 to 12, 1 to 10, 2 to 8, 6 to 8, or 4 to 6. In some embodiments, y is, independently for each occurrence, an integer selected from 7 and 8. In some embodiments, y is, independently for each occurrence, an integer selected from 0, 1, 2, 3, 4, 5, 6, 7, 8, 9, 10, 11, 12, 13, 14, 15, 16, 17, 18, 19, and 20. In certain embodiments, for at least one chain residue, y is an integer selected from 7 and 8. In some embodiments, for at least one chain residue, y is an integer selected from 0 to 6, or 1 and 2. In certain embodiments, y is, independently for each occurrence, an integer selected from 1 to 6, or 1 and 2.

In some embodiments, x+y is, independently for each chain, an integer selected from 0 to 40, 0 to 20, 10 to 20, or 12 to 18. In some embodiments, x+y is, independently for each chain, an integer selected from 13 to 15. In some embodiments, x+y is 15. In some embodiments, x+y is, independently for each chain, an integer selected from 6, 7, 8, 9, 10, 11, 12, 13, 14, 15, 16, 17, 18, 19, 20, 21, 22, 23, and 24.

In some embodiments, the estolide compound of Formula I, II, or III may comprise any number of fatty acid residues to form an “n-mer” estolide. For example, the estolide may be in its dimer (n=0), trimer (n=1), tetramer (n=2), pentamer (n=3), hexamer (n=4), heptamer (n=5), octamer (n=6), nonamer (n=7), or decamer (n=8) form. In some embodiments, n is an integer selected from 0 to 20, 0 to 18, 0 to 16, 0 to 14, 0 to 12, 0 to 10, 0 to 8, or 0 to 6. In some embodiments, n is an integer selected from 0 to 4. In some embodiments, n is 0 or greater than 0. In some embodiments, n is 1, wherein said at least one compound of Formula I, II, or III comprises the trimer. In some embodiments, n is greater than 1. In some embodiments, n is an integer selected from 0, 1, 2, 3, 4, 5, 6, 7, 8, 9, 10, 11, 12, 13, 14, 15, 16, 17, 18, 19, and 20.

In some embodiments, R₁ of Formula I, II, or III is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₁ is selected from C₇ alkyl, C₉ alkyl, C₁₁ alkyl, C₁₃ alkyl, C₁₅ alkyl, and C₁₇ alkyl. In some embodiments, R₁ is selected from C₁₃ to C₁₇ alkyl, such as from C₁₃ alkyl, C₁₅ alkyl, and C₁₇ alkyl. In some embodiments, R₁ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₂ of Formula I, II, or III is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₂ is selected from C₇ alkyl, C₉ alkyl, C₁₁ alkyl, C₁₃ alkyl, C₁₅ alkyl, and C₁₇ alkyl. In some embodiments, R₂ is selected from C₁₃ to C₁₇ alkyl, such as from C₁₃ alkyl, C₁₅ alkyl, and C₁₇ alkyl. In some embodiments, R₂ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₃ is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₃ is selected from C₇ alkyl, C₉ alkyl, C₁₁ alkyl, C₁₃ alkyl, C₁₅ alkyl, and C₁₇ alkyl. In some embodiments, R₃ is selected from C₁₃ to C₁₇ alkyl, such as from C₁₃ alkyl, C₁₅ alkyl, and C₁₇ alkyl. In some embodiments, R₃ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

In some embodiments, R₄ is an optionally substituted alkyl that is saturated or unsaturated, and branched or unbranched. In some embodiments, the alkyl group is a C₁ to C₄₀ alkyl, C₁ to C₂₂ alkyl or C₁ to C₁₈ alkyl. In some embodiments, the alkyl group is selected from C₇ to C₁₇ alkyl. In some embodiments, R₄ is selected from C₇ alkyl, C₉ alkyl, C₁₁ alkyl, C₁₃ alkyl, C₁₅ alkyl, and C₁₇ alkyl. In some embodiments, R₄ is selected from C₁₃ to C₁₇ alkyl, such as from C₁₃ alkyl, C₁₅ alkyl, and C₁₇ alkyl. In some embodiments, R₄ is a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, or C₂₂ alkyl.

As noted above, in certain embodiments, it may be possible to manipulate one or more of the estolides' properties by altering the length of R₁ and/or its degree of saturation. However, in certain embodiments, the level of substitution on R₁ may also be altered to change or even improve the estolides' properties. Without being bound to any particular theory, in certain embodiments, it is believed that the presence of polar substituents on R₁, such as one or more hydroxy groups, may increase the viscosity of the estolide, while increasing melting point. Accordingly, in some embodiments, R₁ will be unsubstituted or optionally substituted with a group that is not hydroxyl.

In some embodiments, the estolide is in its free-acid form, wherein R₂ of Formula I, II, or III is hydrogen. In some embodiments, R₂is selected from optionally substituted alkyl that is saturated or unsaturated, and branched or unbranched. In certain embodiments, the R₂ residue may comprise any desired alkyl group, such as those derived from esterification of the estolide with the alcohols identified in the examples herein. In some embodiments, the alkyl group is selected from C₁ to C₄₀, C₁ to C₂₂, C₃ to C₂₀, C₁ to C₁₈, or C₆ to C₁₂ alkyl. In some embodiments, R₂ may be selected from C₃ alkyl, C₄ alkyl, C₈ alkyl, C₁₂ alkyl, C₁₆ alkyl, C₁₈ alkyl, and C₂₀ alkyl. For example, in certain embodiments, R₂ may be branched, such as isopropyl, isobutyl, or 2-ethylhexyl. In some embodiments, R₂ may be a larger alkyl group, branched or unbranched, comprising C₁₂ alkyl, C₁₆ alkyl, C₁₈ alkyl, or C₂₀ alkyl. Such groups at the R₂ position may be derived from esterification of the free-acid estolide using the Jarcol™ line of alcohols marketed by Jarchem Industries, Inc. of Newark, N.J., including Jarcol™ I-18CG, I-20, I-12, I-16, I-18T, and 85BJ. In some cases, R₂ may be sourced from certain alcohols to provide branched alkyls such as isostearyl and isopalmityl. It should be understood that such isopalmityl and isostearyl akyl groups may cover any branched variation of C₁₆ and C₁₈, respectively. For example, the estolides described herein may comprise highly-branched isopalmityl or isostearyl groups at the R₂ position, derived from the Fineoxocol® line of isopalmityl and isostearyl alcohols marketed by Nissan Chemical America Corporation of Houston, Tex., including Fineoxocol® 180, 180N, and 1600. Without being bound to any particular theory, in certain embodiments, large, highly-branched alkyl groups (e.g., isopalmityl and isostearyl) at the R₂ position of the estolides can provide at least one way to increase an estolide-containing composition's viscosity, while substantially retaining or even reducing its melting point.

In some embodiments, the compounds described herein may comprise a mixture of two or more estolide compounds of Formula I, II, and III. It is possible to characterize the chemical makeup of an estolide, a mixture of estolides, or a composition comprising estolides, by using the compound's, mixture's, or composition's measured estolide number (EN) of compound or composition. The EN represents the average number of fatty acids added to the base fatty acid. The EN also represents the average number of estolide linkages per molecule:

EN=n+1

wherein n is the number of secondary (β) fatty acids. Accordingly, a single estolide compound will have an EN that is a whole number, for example for dimers, trimers, and tetramers:

dimer EN=1

trimer EN=2

tetramer EN=3

However, a composition comprising two or more estolide compounds may have an EN that is a whole number or a fraction of a whole number. For example, a composition having a 1:1 molar ratio of dimer and trimer would have an EN of 1.5, while a composition having a 1:1 molar ratio of tetramer and trimer would have an EN of 2.5.

In some embodiments, the compositions may comprise a mixture of two or more estolides having an EN that is an integer or fraction of an integer that is greater than 4.5, or even 5.0. In some embodiments, the EN may be an integer or fraction of an integer selected from about 1.0 to about 5.0. In some embodiments, the EN is an integer or fraction of an integer selected from 1.2 to about 4.5. In some embodiments, the EN is selected from a value greater than 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6 and 5.8. In some embodiments, the EN is selected from a value less than 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, and 5.0, 5.2, 5.4, 5.6, 5.8, and 6.0. In some embodiments, the EN is selected from 1, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, and 6.0.

As noted above, it should be understood that the chains of the estolide compounds may be independently optionally substituted, wherein one or more hydrogens are removed and replaced with one or more of the substituents identified herein. Similarly, two or more of the hydrogen residues may be removed to provide one or more sites of unsaturation, such as a cis or trans double bond. Further, the chains may optionally comprise branched hydrocarbon residues. For example, in some embodiments the estolides described herein may comprise at least one compound of Formula II:

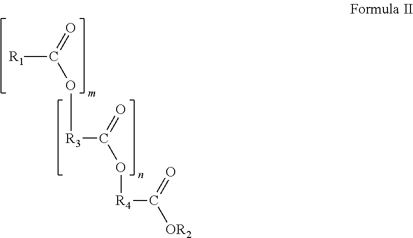

wherein

m is an integer equal to or greater than 1;

n is an integer equal to or greater than 0;

R₁, independently for each occurrence, is an optionally substituted alkyl that is saturated or unsaturated, and branched or unbranched;

R₂ is selected from hydrogen and optionally substituted alkyl that is saturated or unsaturated, and branched or unbranched; and

R₃ and R₄, independently for each occurrence, are selected from optionally substituted alkyl that is saturated or unsaturated, and branched or unbranched.

In certain embodiments, m is 1. In some embodiments, m is an integer selected from 2, 3, 4, and 5. In some embodiments, n is an integer selected from 1, 2, 3, 4, 5, 6, 7, 8, 9, 10, 11, and 12. In some embodiments, one or more R₃ differs from one or more other R₃ in a compound of Formula II. In some embodiments, one or more R₃ differs from R₄ in a compound of Formula II. In some embodiments, if the compounds of Formula II are prepared from one or more polyunsaturated fatty acids, it is possible that one or more of R₃ and R₄ will have one or more sites of unsaturation. In some embodiments, if the compounds of Formula II are prepared from one or more branched fatty acids, it is possible that one or more of R₃ and R₄ will be branched.

In some embodiments, R₃ and R₄ can be CH₃(CH₂)_{y}CH(CH₂)ₓ—, where x is, independently for each occurrence, an integer selected from 0, 1, 2, 3, 4, 5, 6, 7, 8, 9, 10, 11, 12, 13, 14, 15, 16, 17, 18, 19, and 20, and y is, independently for each occurrence, an integer selected from 0, 1, 2, 3, 4, 5, 6, 7, 8, 9, 10, 11, 12, 13, 14, 15, 16, 17, 18, 19, and 20. Where both R₃ and R₄ are CH₃(CH₂)_{y}CH(CH₂)ₓ—, the compounds may be compounds according to Formula I and III.

Without being bound to any particular theory, in certain embodiments, altering the EN produces estolide-containing compositions having desired viscometric properties while substantially retaining or even reducing melting point. For example, in some embodiments the estolides exhibit a decreased melting point upon increasing the EN value. Accordingly, in certain embodiments, a method is provided for retaining or decreasing the melting point of an estolide base oil by increasing the EN of the base oil, or a method is provided for retaining or decreasing the melting point of a composition comprising an estolide base oil by increasing the EN of the base oil. In some embodiments, the method comprises: selecting an estolide base oil having an initial EN and an initial melting point; and removing at least a portion of the base oil, said portion exhibiting an EN that is less than the initial EN of the base oil, wherein the resulting estolide base oil exhibits an EN that is greater than the initial EN of the base oil, and a melting point that is equal to or lower than the initial melting point of the base oil. In some embodiments, the selected estolide base oil is prepared by oligomerizing at least one first unsaturated fatty acid with at least one second unsaturated fatty acid and/or saturated fatty acid. In some embodiments, the removing at least a portion of the base oil or a composition comprising two or more estolide compounds is accomplished by use of at least one of distillation, chromatography, membrane separation, phase separation, affinity separation, and solvent extraction. In some embodiments, the distillation takes place at a temperature and/or pressure that is suitable to separate the estolide base oil or a composition comprising two or more estolide compounds into different “cuts” that individually exhibit different EN values. In some embodiments, this may be accomplished by subjecting the base oil or a composition comprising two or more estolide compounds to a temperature of at least about 250° C. and an absolute pressure of no greater than about 25 microns. In some embodiments, the distillation takes place at a temperature range of about 250° C. to about 310° C. and an absolute pressure range of about 10 microns to about 25 microns.

In some embodiments, estolide compounds and compositions exhibit an EN that is greater than or equal to 1, such as an integer or fraction of an integer selected from about 1.0 to about 2.0. In some embodiments, the EN is an integer or fraction of an integer selected from about 1.0 to about 1.6. In some embodiments, the EN is a fraction of an integer selected from about 1.1 to about 1.5. In some embodiments, the EN is selected from a value greater than 1.0, 1.1, 1.2, 1.3, 1.4, 1.5, 1.6, 1.7, 1.8, and 1.9. In some embodiments, the EN is selected from a value less than 1.1, 1.2, 1.3, 1.4, 1.5, 1.6, 1.7, 1.8, 1.9, and 2.0.

In some embodiments, the EN is greater than or equal to 1.5, such as an integer or fraction of an integer selected from about 1.8 to about 2.8. In some embodiments, the EN is an integer or fraction of an integer selected from about 2.0 to about 2.6. In some embodiments, the EN is a fraction of an integer selected from about 2.1 to about 2.5. In some embodiments, the EN is selected from a value greater than 1.8, 1.9, 2.0, 2.1, 2.2, 2.3, 2.4, 2.5, 2.6, and 2.7. In some embodiments, the EN is selected from a value less than 1.9, 2.0, 2.1, 2.2, 2.3, 2.4, 2.5, 2.6, 2.7, and 2.8. In some embodiments, the EN is about 1.8, 2.0, 2.2, 2.4, 2.6, or 2.8.

In some embodiments, the EN is greater than or equal to about 4, such as an integer or fraction of an integer selected from about 4.0 to about 5.0. In some embodiments, the EN is a fraction of an integer selected from about 4.2 to about 4.8. In some embodiments, the EN is a fraction of an integer selected from about 4.3 to about 4.7. In some embodiments, the EN is selected from a value greater than 4.0, 4.1, 4.2, 4.3, 4.4, 4.5, 4.6, 4.7, 4.8, and 4.9. In some embodiments, the EN is selected from a value less than 4.1, 4.2, 4.3, 4.4, 4.5, 4.6, 4.7, 4.8, 4.9, and 5.0. In some embodiments, the EN is about 4.0, 4.2, 4.4, 4.6, 4.8, or 5.0.

In some embodiments, the EN is greater than or equal to about 5, such as an integer or fraction of an integer selected from about 5.0 to about 6.0. In some embodiments, the EN is a fraction of an integer selected from about 5.2 to about 5.8. In some embodiments, the EN is a fraction of an integer selected from about 5.3 to about 5.7. In some embodiments, the EN is selected from a value greater than 5.0, 5.1, 5.2, 5.3, 5.4, 5.5, 5.6, 5.7, 5.8, and 5.9. In some embodiments, the EN is selected from a value less than 5.1, 5.2, 5.3, 5.4, 5.5, 5.6, 5.7, 5.8, 5.9, and 6.0. In some embodiments, the EN is about 5.0, 5.2, 5.4, 5.4, 5.6, 5.8, or 6.0.

In some embodiments, the EN is greater than or equal to 1, such as an integer or fraction of an integer selected from about 1.0 to about 2.0. In some embodiments, the EN is a fraction of an integer selected from about 1.1 to about 1.7. In some embodiments, the EN is a fraction of an integer selected from about 1.1 to about 1.5. In some embodiments, the EN is selected from a value greater than 1.0, 1.1, 1.2, 1.3, 1.4, 1.5, 1.6, 1.7, 1.8, or 1.9. In some embodiments, the EN is selected from a value less than 1.2, 1.3, 1.4, 1.5, 1.6, 1.7, 1.8, 1.9, or 2.0. In some embodiments, the EN is about 1.0, 1.2, 1.4, 1.6, 1.8, or 2.0. In some embodiments, the EN is greater than or equal to 1, such as an integer or fraction of an integer selected from about 1.2 to about 2.2. In some embodiments, the EN is an integer or fraction of an integer selected from about 1.4 to about 2.0. In some embodiments, the EN is a fraction of an integer selected from about 1.5 to about 1.9. In some embodiments, the EN is selected from a value greater than 1.0, 1.1. 1.2, 1.3, 1.4, 1.5, 1.6, 1.7, 1.8, 1.9, 2.0, and 2.1. In some embodiments, the EN is selected from a value less than 1.2, 1.3, 1.4, 1.5, 1.6, 1.7, 1.8, 1.9, 2.0, 2.1, and 2.2. In some embodiments, the EN is about 1.0, 1.2, 1.4, 1.6, 1.8, 2.0, or 2.2.

In some embodiments, the EN is greater than or equal to 2, such as an integer or fraction of an integer selected from about 2.8 to about 3.8. In some embodiments, the EN is an integer or fraction of an integer selected from about 2.9 to about 3.5. In some embodiments, the EN is an integer or fraction of an integer selected from about 3.0 to about 3.4. In some embodiments, the EN is selected from a value greater than 2.0, 2.1, 2.2., 2.4, 2.5, 2.6, 2.7, 2.8, 2.9, 3.0, 3.1, 3.4, 3.5, 3.6, and 3.7. In some embodiments, the EN is selected from a value less than 2.2, 2.3, 2.4, 2.5, 2.6, 2.7, 2.8, 2.9, 3.0, 3.1, 3.2, 3.3, 3.4, 3.5, 3.6, 3.7, and 3.8. In some embodiments, the EN is about 2.0, 2.2, 2.4, 2.6, 2.8, 3.0, 3.2, 3.4, 3.6, or 3.8.

Typically, base stocks and estolide-containing compositions exhibit certain lubricity, viscosity, and/or melting point characteristics. For example, in certain embodiments, the base oils, compounds, and compositions may exhibit viscosities that range from about 10 cSt to about 250 cSt at 40° C., and/or about 3 cSt to about 30 cSt at 100° C. In some embodiments, the base oils, compounds, and compositions may exhibit viscosities within a range from about 50 cSt to about 150 cSt at 40° C., and/or about 10 cSt to about 20 cSt at 100° C.

In some embodiments, the estolide compounds and compositions may exhibit viscosities less than about 55 cSt at 40° C. or less than about 45 cSt at 40° C., and/or less than about 12 cSt at 100° C. or less than about 10 cSt at 100° C. In some embodiments, the estolide compounds and compositions may exhibit viscosities within a range from about 25 cSt to about 55 cSt at 40° C., and/or about 5 cSt to about 11 cSt at 100° C. In some embodiments, the estolide compounds and compositions may exhibit viscosities within a range from about 35 cSt to about 45 cSt at 40° C., and/or about 6 cSt to about 10 cSt at 100° C. In some embodiments, the estolide compounds and compositions may exhibit viscosities within a range from about 38 cSt to about 43 cSt at 40° C., and/or about 7 cSt to about 9 cSt at 100° C.

In some embodiments, the estolide compounds and compositions may exhibit viscosities less than about 120 cSt at 40° C. or less than about 100 cSt at 40° C., and/or less than about 18 cSt at 100° C. or less than about 17 cSt at 100° C. In some embodiments, the estolide compounds and compositions may exhibit a viscosity within a range from about 70 cSt to about 120 cSt at 40° C., and/or about 12 cSt to about 18 cSt at 100° C. In some embodiments, the estolide compounds and compositions may exhibit viscosities within a range from about 80 cSt to about 100 cSt at 40° C., and/or about 13 cSt to about 17 cSt at 100° C. In some embodiments, the estolide compounds and compositions may exhibit viscosities within a range from about 85 cSt to about 95 cSt at 40° C., and/or about 14 cSt to about 16 cSt at 100° C.

In some embodiments, the estolide compounds and compositions may exhibit viscosities greater than about 180 cSt at 40° C. or greater than about 200 cSt at 40° C., and/or greater than about 20 cSt at 100° C. or greater than about 25 cSt at 100° C. In some embodiments, the estolide compounds and compositions may exhibit a viscosity within a range from about 180 cSt to about 230 cSt at 40° C., and/or about 25 cSt to about 31 cSt at 100° C. In some embodiments, the estolide compounds and compositions may exhibit viscosities within a range from about 200 cSt to about 250 cSt at 40° C., and/or about 25 cSt to about 35 cSt at 100° C. In some embodiments, the estolide compounds and compositions may exhibit viscosities within a range from about 210 cSt to about 230 cSt at 40° C., and/or about 28 cSt to about 33 cSt at 100° C. In some embodiments, the estolide compounds and compositions may exhibit viscosities within a range from about 200 cSt to about 220 cSt at 40° C., and/or about 26 cSt to about 30 cSt at 100° C. In some embodiments, the estolide compounds and compositions may exhibit viscosities within a range from about 205 cSt to about 215 cSt at 40° C., and/or about 27 cSt to about 29 cSt at 100° C.

In some embodiments, the estolide compounds and compositions may exhibit viscosities less than about 45 cSt at 40° C. or less than about 38 cSt at 40° C., and/or less than about 10 cSt at 100° C. or less than about 9 cSt at 100° C. In some embodiments, the estolide compounds and compositions may exhibit a viscosity within a range from about 20 cSt to about 45 cSt at 40° C., and/or about 4 cSt to about 10 cSt at 100° C. In some embodiments, the estolide compounds and compositions may exhibit viscosities within a range from about 28 cSt to about 38 cSt at 40° C., and/or about 5 cSt to about 9 cSt at 100° C. In some embodiments, the estolide compounds and compositions may exhibit viscosities within a range from about 30 cSt to about 35 cSt at 40° C., and/or about 6 cSt to about 8 cSt at 100° C.

In some embodiments, the estolide compounds and compositions may exhibit viscosities less than about 80 cSt at 40° C. or less than about 70 cSt at 40° C., and/or less than about 14 cSt at 100° C. or less than about 13 cSt at 100° C. In some embodiments, the estolide compounds and compositions may exhibit a viscosity within a range from about 50 cSt to about 80 cSt at 40° C., and/or about 8 cSt to about 14 cSt at 100° C. In some embodiments, the estolide compounds and compositions may exhibit viscosities within a range from about 60 cSt to about 70 cSt at 40° C., and/or about 9 cSt to about 13 cSt at 100° C. In some embodiments, the estolide compounds and compositions may exhibit viscosities within a range from about 63 cSt to about 68 cSt at 40° C., and/or about 10 cSt to about 12 cSt at 100° C.

In some embodiments, the estolide compounds and compositions may exhibit viscosities greater than about 120 cSt at 40° C. or greater than about 130 cSt at 40° C., and/or greater than about 15 cSt at 100° C. or greater than about 18 cSt at 100° C. In some embodiments, the estolide compounds and compositions may exhibit a viscosity within a range from about 120 cSt to about 150 cSt at 40° C., and/or about 16 cSt to about 24 cSt at 100° C. In some embodiments, the estolide compounds and compositions may exhibit viscosities within a range from about 130 cSt to about 160 cSt at 40° C., and/or about 17 cSt to about 28 cSt at 100 ° C. In some embodiments, the estolide compounds and compositions may exhibit viscosities within a range from about 130 cSt to about 145 cSt at 40° C., and/or about 17 cSt to about 23 cSt at 100° C. In some embodiments, the estolide compounds and compositions may exhibit viscosities within a range from about 135 cSt to about 140 cSt at 40° C., and/or about 19 cSt to about 21 cSt at 100° C. In some embodiments, the estolide compounds and compositions may exhibit viscosities of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, or 400 cSt. at 40° C. In some embodiments, the estolide compounds and compositions may exhibit viscosities of about 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30 cSt at 100° C.

In some embodiments, the estolide compounds and compositions may exhibit viscosities less than about 200, 250, 300, 350, 400, 450, 500, or 550 cSt at 0° C. In some embodiments, the estolide compounds and compositions may exhibit a viscosity within a range from about 200 cSt to about 250 cSt at 0° C. In some embodiments, the estolide compounds and compositions may exhibit a viscosity within a range from about 250 cSt to about 300 cSt at 0° C. In some embodiments, the estolide compounds and compositions may exhibit a viscosity within a range from about 300 cSt to about 350 cSt at 0° C. In some embodiments, the estolide compounds and compositions may exhibit a viscosity within a range from about 350 cSt to about 400 cSt at 0° C. In some embodiments, the estolide compounds and compositions may exhibit a viscosity within a range from about 400 cSt to about 450 cSt at 0° C. In some embodiments, the estolide compounds and compositions may exhibit a viscosity within a range from about 450 cSt to about 500 cSt at 0° C. In some embodiments, the estolide compounds and compositions may exhibit a viscosity within a range from about 500 cSt to about 550 cSt at 0° C. In some embodiments, the estolide compounds and compositions may exhibit viscosities of about 100, 125, 150, 175, 200, 225, 250, 275, 300, 325, 350, 375, 400, 425, 450, 475, 500, 525, or 550 cSt at 0° C.

In some embodiments, estolide compounds and compositions may exhibit desirable low-temperature melting point properties. In some embodiments, the estolide compounds and compositions may exhibit a melting point lower than about −20° C., about −25° C., about −35° C., −40° C., or even about −50° C. In some embodiments, the estolide compounds and compositions have a melting point of about −25° C. to about −45° C. In some embodiments, the melting point falls within a range of about −30° C. to about −40° C., about −34° C. to about −38° C., about −30° C. to about −45° C., −35° C. to about −45° C., 34° C. to about −42° C., about −38° C. to about −42° C., or about 36° C. to about −40° C. In some embodiments, the melting point falls within the range of about −27° C. to about −37° C., or about −30° C. to about −34° C. In some embodiments, the melting point falls within the range of about −25° C. to about −35° C., or about −28° C. to about −32° C. In some embodiments, the melting point falls within the range of about −28° C. to about −38° C., or about −31° C. to about −35° C. In some embodiments, the melting point falls within the range of about −31° C. to about −41° C., or about −34° C. to about −38° C. In some embodiments, the melting point falls within the range of about −40° C. to about −50° C., or about −42° C. to about −48° C. In some embodiments, the melting point falls within the range of about −50° C. to about −60° C., or about −52° C. to about −58° C. In some embodiments, the upper bound of the melting point is less than about - 35° C., about −36° C., about −37° C., about −38° C., about −39° C., about −40° C., about −41° C., about −42° C., about −43° C., about −44° C., or about −45° C. In some embodiments, the lower bound of the melting point is greater than about −70° C., about −69° C., about −68° C., about −67° C., about −66° C., about −65° C., about −64° C., about −63° C., about −62° C., about −61° C., about −60° C., about −59° C., about −58° C., about −57° C., about −56° C., −55° C., about −54° C., about −53° C., about −52° C., −51, about −50° C., about −49° C., about −48° C., about −47° C., about −46° C., or about −45° C.

In addition, in certain embodiments, the estolides may exhibit decreased Iodine Values (IV) when compared to estolides prepared by other methods. IV is a measure of the degree of total unsaturation of an oil, and is determined by measuring the amount of iodine per gram of estolide (cg/g). In certain instances, oils having a higher degree of unsaturation may be more susceptible to creating corrosiveness and deposits, and may exhibit lower levels of oxidative stability. Compounds having a higher degree of unsaturation will have more points of unsaturation for iodine to react with, resulting in a higher IV. Thus, in certain embodiments, it may be desirable to reduce the IV of estolides in an effort to increase the oil's oxidative stability, while also decreasing harmful deposits and the corrosiveness of the oil.

In some embodiments, estolide compounds and compositions described herein have an IV of less than about 40 cg/g or less than about 35 cg/g. In some embodiments, estolides have an IV of less than about 30 cg/g, less than about 25 cg/g, less than about 20 cg/g, less than about 15 cg/g, less than about 10 cg/g, or less than about 5 cg/g. In some embodiments, estolides have an IV of about 0 cg/g. The IV of a composition may be reduced by decreasing the estolide's degree of unsaturation. This may be accomplished by, for example, by increasing the amount of saturated capping materials relative to unsaturated capping materials when synthesizing the estolides. Alternatively, in certain embodiments, IV may be reduced by hydrogenating estolides having unsaturated caps.

In certain embodiments, the composition has a kinematic viscosity essentially the same as the kinematic viscosity for the estolide base oil included in the composition. In certain embodiments, the composition has a kinematic viscosity within approximately 1% or approximately 2% of the kinematic viscosity of the estolide base oil included within the composition. In certain embodiments, the composition has a kinematic viscosity within 0.2%, 0.4%, 0.6%, 0.8%, 1.0%, 1.2%, 1.4%, 1.6%, 1.8%, or 2% of the kinematic viscosity of the estolide estolide base oil included in the composition. In certain embodiments, the composition has a kinematic viscosity that is less than or equal to about 15 cSt at 100° C. In certain embodiments, the composition has a kinematic viscosity that is less than or equal to about 50 cSt at 40° C. In certain embodiments, the composition has a kinematic viscosity that is less than or equal to about 500 cSt at 0° C.

In certain embodiments, the estolide base oil has a total acid number equal to or less than about 0.5, 0.4, 0.3, 0.2, or even 0.1 mg KOH/g. In certain embodiments, the estolide base oil has a total acid number of less than about 0.1 mg KOH/g, such as about 0.05 to about 0.1 mg KOH/g. In certain embodiments, the estolide base oil has a total acid number equal to or less than about 0.05 mg KOH/g. In certain embodiments, the estolide base oil has a total acid number of about 0.02 to about 0.06 mg KOH/g. In certain embodiments, the estolide base oil has a total acid number of about 0, 0.01, 0.02, 0.03, 0.04, 0.05, 0.06, 0.07, 0.08, 0.09, or 0.1 mg KOH/g. In certain embodiments, the composition has a total acid number essentially the same as the total acid number for the estolide base oil included in the composition.

In certain embodiments, the compositions described herein comprise or consist essentially of an estolide base oil, wherein said base oil comprises at least one compound of Formulas I, II, and/or III.

As discussed above, the estolides described herein may have improved properties which render them useful personal care and cosmetic formulations. Exemplary personal care and cosmetic products include but are not limited to a shampoo, conditioner, hair lotion, tonic, hair spray, hair mousse, hair gel, hair dyes, moisturizer, suntan lotion, color cosmetic, body lotion, hand cream, baby skin-care product, facial cream, lipstick, lip balm, mascara, blush, eyeliner, nail products, baby shampoo, baby moisturizer, baby lotion, shower gel, soap, shaving product, deodorant, bath cream, body wash, serum, cream, solid, gel, lubricant, gelly, balm, tooth paste, whitening gel, disposable towel, disposable wipe or ointment.

In certain embodiments, the estolide compounds described herein provide a level of control over viscosity and consistency factors in many aqueous- and oil-based systems where control over the rheology is a concern. Embodiments may include cosmetic and personal care applications including hair styling, hair conditioners, shampoos, bath preparations, cosmetic creams, gels, lotions, ointments, deodorants, powders, skin cleansers, skin conditioners, skin emollients, skin moisturizers, skin wipes, sunscreens, shaving preparations, and fabric softeners, wherein the estolide compounds may help to provide desirable gel strength and shear thinning characteristics, and versatile viscometric properties and synergistic interactions with added agents to adjust their rheology profile to optimize properties such as sedimentation, flow and leveling, sagging, and spattering.

For hair care products, in certain embodiments, the estolide compounds provide one or more of: improvements in intra-fiber moisture retention and protection from thermal damage; reduce coefficient of friction of hair to prevent mechanical damage; provide protection from thermal treatments; provide anti-breakage benefits; strengthen hair fibers; reduce static build-up; improve elasticity; and increase shine. For skin care products, in certain embodiments, the estolide compounds provide one or more of: improved elasticity; moisture retention; hydrating/moisturizing properties; and anti-aging properties.

In certain embodiments, the amount of estolides incorporated into a shampoo or conditioner varies, but is provided in an amount effective to enhance the performance of the shampoo or conditioner. An effective amount is defined herein as that concentration which is effective to improve one or more of rinseability, wet feel, detangling, dry comb feel, style management, shine, or body of washed hair, relative to control shampoo lacking estolides. Suitable concentrations may be readily determined by routine experimentation and will vary with the specific shampoo or conditioner formulation. In certain embodiments, suitable concentrations of estolides in the shampoo or conditioner may be between about 1 to about 20% by weight, about 1 to about 10%, or about 2 to about 8%. In certain embodiments, the balance of the shampoo or conditioner is prepared and formulated using conventional components or agents and water.

In certain embodiments, the shampoo will include at least one surfactant, which may be used as a cleansing agent. In certain embodiments, the shampoo will include a thickener or viscosity modifier. A number of exemplary surfactants have been previously described as cleansing agents in shampoos and may be suitable for use herein, which include, for example, anionic, nonionic, amphoteric, and zwitterionic surfactants, or mixtures thereof. In certain embodiments, the concentration of the surfactant is present in amounts effective to be capable of cleaning hair. In certain embodiments, suitable concentrations include between about 1% to about 70% by weight, or from about 10% to about 50%. In certain embodiments, the thickener present in an amount effective to assist in the hand application of the shampoo.

Ammonium lauryl ether sulfate and coconut diethanolamide (DEA) are exemplary surfactants for use in shampoos. Specific examples of other suitable anionic surfactants includes sodium lauryl sulfate, sodium lauryl ether sulfate, ammonium lauryl sulfate, triethanolamine lauryl sulfate, sodium C₁₄-C₁₆ olefin sulfonate, ammonium C₁₂-C₁₅ pareth sulfate, sodium myristyl ether sulfate, disodium monooleamidosulfosuccinate, ammonium lauryl sulfosuccinate, sodium dodecylbenzene sulfonate, triethanolamine dodecylbenzene sulfonate, and sodium N-lauryol sarcosinate. Examples of amphoteric surfactants include cocoamphocarboxyglycinate, cocoamphocarboxypropionate, cocobetaine, N-cocamidopropyldimethylglycine, N-lauryl-N-carboxymethyl-N-(2-hydroxyethyl)ethylenediamine; betaines such as alpha-(tetradecyldimethylammonio)acetate, beta-(hexadecyldiethylammonio)propionate, and gamma-(dodecyldimethylammonio)butyrate; and sultaines such as 3-(dodecyldimethylammonio)-propane-1-sulfonate, and 3-(tetradecyldimethylammonio)ethane-1-sulfonate. Examples of nonionic surfactants suitable for use may include fatty acid diethanolamides such as isostearic acid DEA, lauric acid DEA, capric acid DEA, linoleic acid DEA, myristic acid DEA, oleic acid DEA, and stearic acid DEA; fatty acid monoethanolamides such as coconut fatty acid monoethanolamide; fatty acid monisopropanolamides such as oleic acid monoisopropanolamide and lauric acid monoisopropanolamide; alkyl amine oxides such as N-cocodimethylamine oxide, N-lauryl dimethylamine oxide, N-myristyl dimethylamine oxide, and N-stearyl dimethylamine oxide; N-acyl amine oxides such as N-cocoamidopropyl dimethylamine oxide and N-tallowamidopropyl dimethylamine oxide; N-alkoxyalkyl amine oxides such as bis(2-hydroxyethyl)C₁₂-C₁₅ alkoxy-propylamine oxide; and polyoxyethylene sorbitol fatty acid esters (e.g., polysorbates 20 and 80). Examples of zwitterionic surfactants which may be used include 4-[N,N-di(2-hydroxyethyl)-N-octadecylammonio]butane-1-carboxylate, 5-[S-3-hydroxypropyl-S-hexadecylsulfonio]-3-hydroxypentane-1-sulfate, 3-[P,P-diethyl-P-3,6,9-trioxatetradexocylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)-propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3- [S-ethyl-S-(3-dodecoxy-2-hydroxypropyl)sulfonio]propane-1-phosphate, 3-[P,P-dimethyl-P-dodecylphosphonio]-propane-1-phosphonate, and 5-[N,N-di(3-hydroxypropyl)-N-hexadecylammonio]-2-hydroxypentane-1-sulfate.

Suitable thickening agents may include one or more of sodium alignate; gum arabic; guar gum; hydroxypropyl guar gum; cellulose derivatives such as methylcellulose, hydroxypropyl methylcellulose, hydroxyethylcellulose, carboxymethylcellulose, and hydroxypropylcellulose; polymer of acrylic acid, such as acrylates/C10-C30 alkyl acrylate crosspolymers, and those crosslinked with an unsaturated polyfunctional agent as a polyallyl ether of sucrose (e.g., carbomers), which may or may not be neutralized with one or more salts; starch and starch derivatives such as hydroxyethylamylose and starch amylose; locust bean gum; electrolytes such as sodium chloride and ammonium chloride; saccharides such as fructose and glucose; derivatives of saccharides such as PEG-120 methyl glucose dioleate; diethanolamides of long chain fatty acids; block polymers of ethylene oxide and propylene oxide such as PLURONIC F88 (BASF Wyandotte); polyvinyl alcohol; and ethyl alcohol.

In certain embodiments, the shampoo also contains an optional component for further improving performance, marketability, or aesthetics, such as one or more of a conditioner/conditioning agent, a foaming agent, a foam stabilizer, a preservative, a chelating agent, an antimicrobial, a fragrance, a colorant, an opacifier, a pearlizing agent, a moisturizing agent, a medicament, a buffer and/or a pH modifier, or a UV absorber.

Exemplary conditioning agents include, but are not limited to, silicones, cationic surfactants and quaternary ammonium compounds, and synthetic cationic polymers. Exemplary silicon conditioning agents include polyalkyl siloxanes such as polydimethyl siloxanes (e.g., dimethicone); polyalkylaryl siloxanes such as polymethylphenylsiloxanes; polyether siloxane copolymers such as polypropylene oxide modified dimethylpolysiloxane; and silicone gums such as polydimethylsiloxane, (polydimethylsiloxane)(methylvinylsiloxane) copolymer, poly(dimethylsiloxane)(diphenyl)(methylvinylsiloxane) copolymer, and mixtures thereof. Other exemplary conditioning agents include but are not limited to cationic surfactants which contain amino or quaternary ammonium hydrophilic moieties in the molecule which are positively charged, such as quaternary ammonium salts. Specific examples include ditallowdimethyl ammonium chloride, ditallowdimethyl ammonium methyl sulfate, hexadecyl trimethyl ammonium chloride, lauryl trimethyl ammonium chloride, trihexadecyl methyl ammonium chloride, dihexadecyl dimethyl ammonium chloride, di(hydrogenated tallow)dimethyl ammonium chloride, dioctadecyl dimethyl ammonium chloride, dieicosyl dimethyl ammonium chloride, didocosyl dimethyl ammonium chloride, di(hydrogenated tallow)dimethyl ammonium acetate, dihexadecyl dimethyl ammonium acetate, ditallow dipropyl ammonium phosphate, ditallow dimethyl ammonium nitrate, di(coconutalkyl)dimethyl ammonium chloride, and stearyl dimethyl benzyl ammonium chloride. Other exemplary cationic conditioning agents include quaternary nitrogen derivatives of cellulose ethers, homopolymers of dimethyldiallyl ammonium chloride, copolymers of acrylamide and dimethyl diallyl ammonium chloride, homopolymers or copolymers derived from acylic acid or methacrylic acid which contain cationic nitrogen functional groups attached to the polymer by ester or amide linkages, polycondensation products of N,N′-bis-(2,3-epoxypropyl)-piperazine or piperazine-bis-acrylamide and piperazine, and copolymers of vinylpyrrolidone and acrylic acid esters with quaternary nitrogen functionality.

Exemplary preservatives include benzyl alcohol, methyl paraben, propyl paraben, formaldehyde, DMDM hydantoin, 5-bromo-5-nitro-1,3-dioxane, sorbic acid, diazolidinyl urea, imidazolidinyl urea, and phenoxyethanol. Exemplary chelating agents include disodium ethylenediamine tetraacetate. Exemplary pearlizing agents include ethylene glycol monostearate and ethylene glycol distearate. Exemplary pH adjusting agents include bases such as sodium hydroxide and sodium carbonate; mineral acids such as hydrochloric acid, sulfuric acid, and phosphoric acid; monocarboxylic acids such as acetic acid, lactic acid, and propionic acid; and polycarboxylic acids such as succinic acid, adipic acid, and citric acid.

In certain embodiments, the conditioning compositions are similar to the shampoo with the exception of the surfactant cleansing agent, which is typically omitted. Thus, in certain embodiments, the conditioner comprises at least one hair conditioning agent, a thickener, water, and at least one estolide. In certain embodiments, these components and their formulation may be the same as described hereinabove. However, in certain embodiments, the amount of water is increased. As with the shampoo, in certain embodiments, the conditioner comprises one or more optional foaming agents, foam stabilizers, preservatives and/or chelating agents, antimicrobials, fragrances, colorants, opacifiers, pearlizing agents, moisturizing agents, medicaments, buffers and/or pH modifiers, and UV absorbers, for further improving performance, marketability, or aesthetics.

In certain embodiments, the shampoos and conditioners described herein can be made using conventional techniques. While mixing the components together with agitation is generally satisfactory, application of gentle heating may aid emulsification in certain embodiments. The pH of the present compositions may vary with the particular surfactant(s) selected. In certain embodiments, the pH ranges between about 4.5 to 8.5 or about 5.5 to 6.0.

In certain embodiments, the estolide-containing compositions described herein may be suitable for use as skin moisturizing and lotion products, such as those formulated into a variety of compositions, including liquid, solid and gel-like, for delivery of its moisturizing benefit. When formulated with a solid, the moisturizing compound can be present with large or small quantities of soap-type compounds and surfactants. When formulated with a liquid or gel composition, the moisturizing compound may be formulated with various amounts of water depending upon the usage of the composition as a cleansing composition, as well as various surfactants of an anionic, nonionic, cationic, amphoteric type, or mixtures thereof, such as those previously described herein. In certain embodiments, the liquid or gel formulations, particularly the liquids, can be formed as a cream or lotion or free flowing liquid which has cleaning abilities, moisturizing and/or conditioning abilities, or a mixture of the cleansing with the moisturizing and/or conditioning benefits. By conditioning is meant increasing the smoothness or suppleness of the skin. By moisturizing is meant the actual increasing of water content of the skin.

In certain embodiments, other conditioning and moisturizing agents also can be present in the compositions described herein. Exemplary moisturizing or conditioning materials include urea, lactic acid, pyrrolidone carboxylic acid, amino acids and salts thereof.

In certain embodiments, the compositions comprise at least one occlusive agent. Occlusive agents may include substances which form on the skin thin films of limited permeability, serving to hold water within the skin and prevent dehydration. In certain embodiments the occlusive agents are hydrophobic oils and waxes. Exemplary occlusive agents include but are not limited to: hydrocarbon oils and waxes such as mineral oil, petrolatum, paraffin, ceresin, ozokenite, microcrystalline wax; silicone oils such as dimethyl polysiloxanes, methylphenyl polysiloxanes, silicone glycol copolymers; triglyceride esters, for example, vegetable and animal fats and oils; glyceride esters and esters such as acetylated monoglycerides, and ethoxylated monoglycerides; alkyl and alkenyl esters of fatty acids having 10 to 20 carbon atoms such as hexyl laurate, isohexyl laurate, isohexyl palmitate, isopropyl myristate, isopropyl palmitate, decyl oleate, isodecyl oleate, hexadecyl stearate, decyl stearate, isopropyl isostearate, diisopropyl adipate, diisohexyl adipate, dihexyl decyl adipate, diisopropyl sebacate, lauryl lactate, myristyl lactate, cetyl lactate, oleyl myristate, oleyl stearate and oleyl oleate; fatty alcohols having 10 to 20 carbon atoms such as lauryl, myristyl, cetyl, hexadecyl, stearyl, isostearyl, hydroxystearyl, oleyl, ricinoleyl, behenyl, erucyl, and 2-octyl dodecanyl alcohols; Lanolin and derivatives including lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols and lanolin alcohols (linoleate are illustrative emollients derived from lanolin); and natural waxes, esters thereof and ethoxylated natural waxes, beeswax, spermaceti, myristyl myristate, stearyl stearate, polyoxyethylene sorbitol beeswax, carnauba wax and candelilla wax.

In certain embodiments the composition comprises one or more humectants. Exemplary humectants include polyols like C₂-C₆ polyols, such as glycerol, sorbitol, propylene glycol, 1,3-butylene glycol, 1,2-hexanediol, and 1,2-octanediol. Exemplary humectants also include polyethylene glycols having molecular weights of from about 100 to about 1500. In certain embodiments, the humectants will not form occlusive films but may cooperate with other materials to form a film having occlusive properties. Accordingly, it may be desirable that humectants are not the sole category of skin emollient agent present.

Examples of surfactants which can be employed in the composition include anionic, nonionic, amphoteric and cationic, such as those previously discussed herein.

The present disclosure further relates to methods of making estolides according to Formula I, II, and III. By way of example, the reaction of an unsaturated fatty acid with an organic acid and the esterification of the resulting free acid estolide are illustrated and discussed in the following Schemes 1 and 2. The particular structural formulas used to illustrate the reactions correspond to those for synthesis of compounds according to Formula I and III; however, the methods apply equally to the synthesis of compounds according to Formula II, with use of compounds having structure corresponding to R₃ and R₄ with a reactive site of unsaturation.

As illustrated below, compound 100 represents an unsaturated fatty acid that may serve as the basis for preparing the estolide compounds described herein.

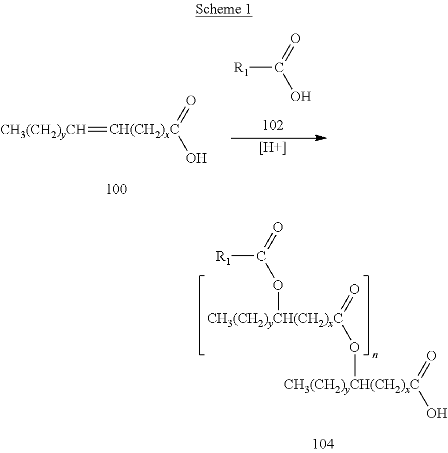

In Scheme 1, wherein x is, independently for each occurrence, an integer selected from 0 to 20, y is, independently for each occurrence, an integer selected from 0 to 20, n is an integer greater than or equal to 1, and R₁ is an optionally substituted alkyl that is saturated or unsaturated, and branched or unbranched, unsaturated fatty acid 100 may be combined with compound 102 and a proton from a proton source to form free acid estolide 104. In certain embodiments, compound 102 is not included, and unsaturated fatty acid 100 may be exposed alone to acidic conditions to form free acid estolide 104, wherein R₁ would represent an unsaturated alkyl group. In certain embodiments, if compound 102 is included in the reaction, R₁ may represent one or more optionally substituted alkyl residues that are saturated or unsaturated and branched or unbranched. Any suitable proton source may be implemented to catalyze the formation of free acid estolide 104, including but not limited to homogenous acids and/or strong acids like hydrochloric acid, sulfuric acid, perchloric acid, nitric acid, triflic acid, and the like.

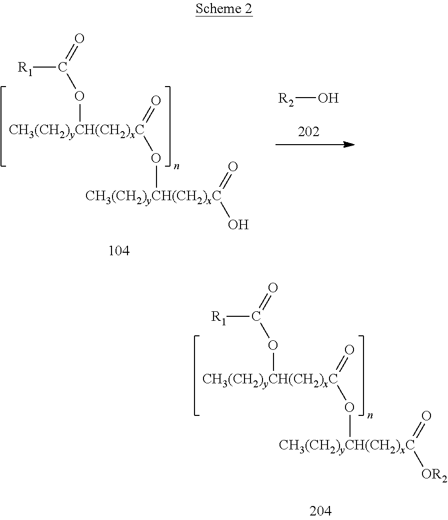

Similarly, in Scheme 2, wherein x is, independently for each occurrence, an integer selected from 0 to 20, y is, independently for each occurrence, an integer selected from 0 to 20, n is an integer greater than or equal to 1, and R₁ and R₂ are each an optionally substituted alkyl that is saturated or unsaturated, and branched or unbranched, free acid estolide 104 may be esterified by any suitable procedure known to those of skilled in the art, such as acid-catalyzed reduction with alcohol 202, to yield esterified estolide 204. Other exemplary methods may include other types of Fischer esterification, such as those using Lewis acid catalysts such as BF₃.

In all of the foregoing examples, the compounds described may be useful alone, as mixtures, or in combination with other compounds, compositions, and/or materials.

Methods for obtaining the novel compounds described herein will be apparent to those of ordinary skill in the art, suitable procedures being described, for example, in the examples below, and in the references cited herein.

### EXAMPLES

### Analytics

Nuclear Magnetic Resonance: NMR spectra were collected using a Bruker Avance 500 spectrometer with an absolute frequency of 500.113 MHz at 300 K using CDCl₃ as the solvent. Chemical shifts were reported as parts per million from tetramethylsilane. The formation of a secondary ester link between fatty acids, indicating the formation of estolide, was verified with ¹H NMR by a peak at about 4.84 ppm.

Estolide Number (EN): The EN was measured by GC analysis. It should be understood that the EN of a composition specifically refers to EN characteristics of any estolide compounds present in the composition. Accordingly, an estolide composition having a particular EN may also comprise other components, such as natural or synthetic additives, other non-estolide base oils, fatty acid esters, e.g., triglycerides, and/or fatty acids, but the EN as used herein, unless otherwise indicated, refers to the value for the estolide fraction of the estolide composition.

Iodine Value (IV): The iodine value is a measure of the degree of total unsaturation of an oil. IV is expressed in terms of centigrams of iodine absorbed per gram of oil sample. Therefore, the higher the iodine value of an oil the higher the level of unsaturation is of that oil. The IV may be measured and/or estimated by GC analysis. Where a composition includes unsaturated compounds other than estolides as set forth in Formula I, II, and III, the estolides can be separated from other unsaturated compounds present in the composition prior to measuring the iodine value of the constituent estolides. For example, if a composition includes unsaturated fatty acids or triglycerides comprising unsaturated fatty acids, these can be separated from the estolides present in the composition prior to measuring the iodine value for the one or more estolides.

Acid Value: The acid value is a measure of the total acid present in an oil. Acid value may be determined by any suitable titration method known to those of ordinary skill in the art. For example, acid values may be determined by the amount of KOH that is required to neutralize a given sample of oil, and thus may be expressed in terms of mg KOH/g of oil.

Gas Chromatography (GC): GC analysis was performed to evaluate the estolide number (EN) and iodine value (IV) of the estolides. This analysis was performed using an Agilent 6890N series gas chromatograph equipped with a flame-ionization detector and an autosampler/injector along with an SP-2380 30 m×0.25 mm i.d. column.

The parameters of the analysis were as follows: column flow at 1.0 mL/min with a helium head pressure of 14.99 psi; split ratio of 50:1; programmed ramp of 120-135° C. at 20° C./min, 135-265° C. at 7° C./min, hold for 5 min at 265° C.; injector and detector temperatures set at 250° C.

Measuring EN and IV by GC: To perform these analyses, the fatty acid components of an estolide sample were reacted with Me0H to form fatty acid methyl esters by a method that left behind a hydroxy group at sites where estolide links were once present. Standards of fatty acid methyl esters were first analyzed to establish elution times.

Sample Preparation: To prepare the samples, 10 mg of estolide was combined with 0.5 mL of 0.5M KOH/MeOH in a vial and heated at 100° C. for 1 hour. This was followed by the addition of 1.5 mL of 1.0 M H₂SO₄/MeOH and heated at 100° C. for 15 minutes and then allowed to cool to room temperature. One (1) mL of H₂O and 1 mL of hexane were then added to the vial and the resulting liquid phases were mixed thoroughly. The layers were then allowed to phase separate for 1 minute. The bottom H₂O layer was removed and discarded. A small amount of drying agent (Na₂SO₄ anhydrous) was then added to the organic layer after which the organic layer was then transferred to a 2 mL crimp cap vial and analyzed.

EN Calculation: The EN is measured as the percent hydroxy fatty acids divided by the percent non-hydroxy fatty acids. As an example, a dimer estolide would result in half of the fatty acids containing a hydroxy functional group, with the other half lacking a hydroxyl functional group. Therefore, the EN would be 50% hydroxy fatty acids divided by 50% non-hydroxy fatty acids, resulting in an EN value of 1 that corresponds to the single estolide link between the capping fatty acid and base fatty acid of the dimer.

IV Calculation: The iodine value is estimated by the following equation based on ASTM Method D97 (ASTM International, Conshohocken, Pa.):

$$IV = \sum 100 \times \frac{{A}_{f} \times {MW}_{I} \times db}{{MW}_{f}}$$

A_{f}=fraction of fatty compound in the sample
MW_{I}=253.81, atomic weight of two iodine atoms added to a double bond
db=number of double bonds on the fatty compound
MW_{f}=molecular weight of the fatty compound

The properties of exemplary estolide compounds and compositions described herein are identified in the following examples and tables.

Other Measurements: Except as otherwise described, melting point is measured by ASTM Method D97-96a, cloud point is measured by ASTM Method D2500, viscosity/kinematic viscosity is measured by ASTM Method D445-97, viscosity index is measured by ASTM Method D2270-93 (Reapproved 1998), specific gravity is measured by ASTM Method D4052, fire point and flash point are measured by ASTM Method D92, evaporative loss is measured by ASTM Method D5800, vapor pressure is measured by ASTM Method D5191, rotating pressure vessel oxidation testing is measured by ASTM Method 2272-11, and acute aqueous toxicity is measured by Organization of Economic Cooperation and Development (OECD) 203.

### Example 1

The acid catalyst reaction was conducted in a 50 gallon Pfaudler RT-Series glass-lined reactor. Oleic acid (65 Kg, OL 700, Twin Rivers) was added to the reactor with 70% perchloric acid (992.3 mL, Aldrich Cat #244252) and heated to 60° C. in vacuo (10 torr abs (Torr absolute; 1 torr=˜1 mmHg)) for 24 hrs while continuously being agitated. After 24 hours the vacuum was released. 2-Ethylhexanol (29.97 Kg) was then added to the reactor and the vacuum was restored. The reaction was allowed to continue under the same conditions (60° C., 10 torr abs) for 4 more hours. At which time, KOH (645.58 g) was dissolved in 90% ethanol/water (5000 mL, 90% EtOH by volume) and added to the reactor to quench the acid. The solution was then allowed to cool for approximately 30 minutes. The contents of the reactor were then pumped through a 1 micron (μ) filter into an accumulator to filter out the salts. Water was then added to the accumulator to wash the oil. The two liquid phases were thoroughly mixed together for approximately 1 hour. The solution was then allowed to phase separate for approximately 30 minutes. The water layer was drained and disposed of. The organic layer was again pumped through a 1μ filter back into the reactor. The reactor was heated to 60° C. in vacuo (10 torr abs) until all ethanol and water ceased to distill from solution. The reactor was then heated to 100° C. in vacuo (10 ton abs) and that temperature was maintained until the 2-ethylhexanol ceased to distill from solution. The remaining material was then distilled using a Myers 15 Centrifugal Distillation still at 200° C. under an absolute pressure of approximately 12 microns (0.012 torr) to remove all monoester material leaving behind estolides (Ex. 1). Certain data are reported below in Tables 1 and 8.

### Example 2

The acid catalyst reaction was conducted in a 50 gallon Pfaudler RT-Series glass-lined reactor. Oleic acid (50 Kg, OL 700, Twin Rivers) and whole cut coconut fatty acid (18.754 Kg, TRC 110, Twin Rivers) were added to the reactor with 70% perchloric acid (1145 mL, Aldrich Cat #244252) and heated to 60° C. in vacuo (10 torr abs) for 24 hrs while continuously being agitated. After 24 hours the vacuum was released. 2-Ethylhexanol (34.58 Kg) was then added to the reactor and the vacuum was restored. The reaction was allowed to continue under the same conditions (60° C., 10 torr abs) for 4 more hours. At which time, KOH (744.9 g) was dissolved in 90% ethanol/water (5000 mL, 90% EtOH by volume) and added to the reactor to quench the acid. The solution was then allowed to cool for approximately 30 minutes. The contents of the reactor were then pumped through a 1μ filter into an accumulator to filter out the salts. Water was then added to the accumulator to wash the oil. The two liquid phases were thoroughly mixed together for approximately 1 hour. The solution was then allowed to phase separate for approximately 30 minutes. The water layer was drained and disposed of. The organic layer was again pumped through a 1μ filter back into the reactor. The reactor was heated to 60° C. in vacuo (10 torr abs) until all ethanol and water ceased to distill from solution. The reactor was then heated to 100° C. in vacuo (10 torr abs) and that temperature was maintained until the 2-ethylhexanol ceased to distill from solution. The remaining material was then distilled using a Myers 15 Centrifugal Distillation still at 200° C. under an absolute pressure of approximately 12 microns (0.012 ton) to remove all monoester material leaving behind estolides (Ex. 2). Certain data are reported below in Tables 2 and 7.

### Example 3

The estolides produced in Example 1 (Ex. 1) were subjected to distillation conditions in a Myers 15 Centrifugal Distillation still at 300° C. under an absolute pressure of approximately 12 microns (0.012 torr). This resulted in a primary distillate having a lower EN average (Ex. 3A), and a distillation residue having a higher EN average (Ex. 3B). Certain data are reported below in Tables 1 and 8.

TABLE 1

| | | Melting | Iodine |
|---|---|---|---|
| Estolide | | Point | Value |
| Base Stock | EN | (° C.) | (cg/g) |
| Ex. 3A | 1.35 | −32 | 31.5 |
| Ex. 1 | 2.34 | −40 | 22.4 |
| Ex. 3B | 4.43 | −40 | 13.8 |

### Example 4

Estolides produced in Example 2 (Ex. 2) were subjected to distillation conditions in a Myers 15 Centrifugal Distillation still at 300° C. under an absolute pressure of approximately 12 microns (0.012 torr). This resulted in a primary distillate having a lower EN average (Ex. 4A), and a distillation residue having a higher EN average (Ex. 4B). Certain data are reported below in Tables 2 and 7.

TABLE 2

| Estolide | | Melting Point | Iodine |
|---|---|---|---|
| Base Stock | EN | (° C.) | Value (cg/g) |
| Ex. 4A | 1.31 | −30 | 13.8 |
| Ex. 2 | 1.82 | −33 | 13.2 |
| Ex. 4B | 3.22 | −36 | 9.0 |

### Example 5

Estolides produced by the method set forth in Example 1 were subjected to distillation conditions (ASTM D-6352) at 1 atm (atmosphere) over the temperature range of about 0° C. to about 710° C., resulting in 10 different estolide cuts recovered at increasing temperatures The amount of material distilled from the sample in each cut and the temperature at which each cut distilled (and recovered) are reported below in Table 3:

TABLE 3

| Cut (% of total) | Temp. (° C.) |
|---|---|
| 1 (1%) | 416.4 |
| 2 (1%) | 418.1 |
| 3 (3%) | 420.7 |
| 4 (20%) | 536.4 |
| 5 (25%) | 553.6 |
| 6 (25%) | 618.6 |
| 7 (20%) | 665.7 |
| 8 (3%) | 687.6 |
| 9 (1%) | 700.6 |
| 10 (1%) | 709.1 |

### Example 6

Estolides made according to the method of Example 2 were subjected to distillation conditions (ASTM D-6352) at 1 atm over the temperature range of about 0° C. to about 730° C., which resulted in 10 different estolide cuts. The amount of each cut and the temperature at which each cut was recovered are reported in Table 4.

TABLE 4

| Cut (% of total) | Temp. (° C.) |
|---|---|
| 1 (1%) | 417.7 |
| 2 (1%) | 420.2 |
| 3 (3%) | 472.0 |
| 4 (5%) | 509.7 |
| 5 (15%) | 533.7 |
| 6 (25%) | 583.4 |
| 7 (25%) | 636.4 |
| 8 (5%) | 655.4 |
| 9 (5%) | 727.0 |
| 10 (15%) | >727.0 |

### Example 7

Estolide base oil 4B (from Example 4) was subjected to distillation conditions (ASTM D-6352) at 1 atm over the temperature range of about 0° C. to about 730° C., which resulted in 9 different estolide cuts. The amount of each cut and the temperature at which each cut was recovered are reported in Table 5a.

TABLE 5a

| Cut (% of total) | Temp. (° C.) |
|---|---|
| 1 (1%) | 432.3 |
| 2 (1%) | 444.0 |
| 3 (3%) | 469.6 |
| 4 (5%) | 521.4 |
| 5 (15%) | 585.4 |
| 6 (25%) | 617.1 |
| 7 (25%) | 675.1 |
| 8 (5%) | 729.9 |
| 9 (20%) | >729.9 |

### Example 8

Estolides were made according to the method set forth in Example 1, except that the 2-ethylhexanol esterifying alcohol used in Example 1 was replaced with various other alcohols. Alcohols used for esterifiction include those identified in Table 5b below. The properties of the resulting estolides are set forth in Table 9.

TABLE 5b

| Alcohol | Structure |
|---|---|
| Jarcol ™ I-18CG | iso-octadecanol |
| Jarcol ™ I-12 | 2-butyloctanol |
| Jarcol ™ I-20 | 2-octyldodecanol |
| Jarcol ™ I-16 | 2-hexyldecanol |
| Jarcol ™ 85BJ | cis-9-octadecen-1-ol |
| Fineoxocol ® 180 | 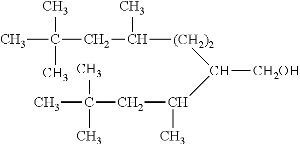 |
| Jarcol ™ I-18T | 2-octyldecanol |

### Example 9

Estolides were made according to the method set forth in Example 2, except the 2ethylhexanol esterifying alcohol was replaced with isobutanol. The properties of the resulting estolides are set forth in Table 9.

### Example 10

Estolides of Formula I, II, and III are prepared according to the method set forth in Examples 1 and 2, except that the 2-ethylhexanol esterifying alcohol is replaced with various other alcohols. Alcohols to be used for esterification include those identified in Table 6 below. Esterifying alcohols to be used, including those listed below, may be saturated or unsaturated, and branched or unbranched, or substituted with one or more alkyl groups selected from methyl, ethyl, propyl, isopropyl, butyl, isobutyl, sec-butyl, tert-butyl, pentyl, isopentyl, neopentyl, hexyl, isohexyl, and the like, to form a branched or unbranched residue at the R₂ position. Examples of combinations of esterifying alcohols and R₂ Substituents are set forth below in Table 6:

TABLE 6

| Alcohol | R₂ Substituents |
|---|---|
| C₁ alkanol | methyl |
| C₂ alkanol | ethyl |
| C₃ alkanol | n-propyl, isopropyl |
| C₄ alkanol | n-butyl, isobutyl, sec-butyl |
| C₅ alkanol | n-pentyl, isopentyl neopentyl |
| C₆ alkanol | n-hexyl, 2-methyl pentyl, 3- |
| | methyl pentyl, 2,2-dimethyl |
| | butyl, 2,3-dimethyl butyl |
| C₇ alkanol | n-heptyl and other structural |
| | isomers |
| C₈ alkanol | n-octyl and other structural |
| | isomers |
| C₉ alkanol | n-nonyl and other structural |
| | isomers |
| C₁₀ alkanol | n-decanyl and other structural |
| | isomers |
| C₁₁ alkanol | n-undecanyl and other structural |
| | isomers |
| C₁₂ alkanol | n-dodecanyl and other structural |
| | isomers |
| C₁₃ alkanol | n-tridecanyl and other structural |
| | isomers |
| C₁₄ alkanol | n-tetradecanyl and other |
| | structural isomers |
| C₁₅ alkanol | n-pentadecanyl and other |
| | structural isomers |
| C₁₆ alkanol | n-hexadecanyl and other |
| | structural isomers |
| C₁₇ alkanol | n-heptadecanyl and other |
| | structural isomers |
| C₁₈ alkanol | n-octadecanyl and other |
| | structural isomers |
| C₁₉ alkanol | n-nonadecanyl and other |
| | structural isomers |
| C₂₀ alkanol | n-icosanyl and other structural |
| | isomers |
| C₂₁ alkanol | n-heneicosanyl and other |
| | structural isomers |
| C₂₂ alkanol | n-docosanyl and other structural |
| | isomers |

TABLE 7

| | | ASTM | | | |
|---|---|---|---|---|---|
| PROPERTY | ADDITIVES | METHOD | Ex. 4A | Ex. 2 | Ex. 4B |
| Color | None | — | Light | Amber | Amber |
| | | | Gold | | |
| Specific Gravity (15.5° C.), g/ml | None | D 4052 | 0.897 | 0.904. | 0.912 |
| Viscosity-Kinematic at 40° C., cSt | None | D 445 | 32.5 | 65.4 | 137.3 |
| Viscosity-Kinematic at 100° C., cSt | None | D 445 | 6.8 | 11.3 | 19.9 |
| Viscosity Index | None | D 2270 | 175 | 167 | 167 |
| Melting Point, ° C. | None | D 97 | −30 | −33 | −36 |
| Cloud Point, ° C. | None | D 2500 | <−30 | <−32 | <−36 |
| Flash Point, ° C. | None | D 92 | 278 | 264 | 284 |
| Fire Point, ° C. | None | D 92 | 300 | 300 | 320 |
| Evaporative Loss (NOACK), wt. % | None | D 5800 | 1.9 | 1.4 | 0.32 |
| Vapor Pressure - Reid (RVP), psi | None | D 5191 | ≈0 | ≈0 | ≈0 |

TABLE 8

| | | ASTM | | | |
|---|---|---|---|---|---|
| PROPERTY | ADDITIVES | METHOD | Ex. 3A | Ex. 1 | Ex. 3B |
| Color | None | — | Light | Amber | Amber |
| | | | Gold | | |
| Specific Gravity (15.5° C.), g/ml | None | D 4052 | 0.897 | 0.906 | 0.917 |
| Viscosity-Kinematic at 40° C., cSt | None | D 445 | 40.9 | 91.2 | 211.6 |
| Viscosity-Kinematic at 100° C., cSt | None | D 445 | 8.0 | 14.8 | 27.8 |
| Viscosity Index | None | D 2270 | 172 | 170 | 169 |
| Melting Point, ° C. | None | D 97 | <−32 | <−40 | <−40 |
| Cloud Point, ° C. | None | D 2500 | −32 | −33 | −40 |
| Flash Point, ° C. | None | D 92 | 278 | 286 | 306 |
| Fire Point, ° C. | None | D 92 | 300 | 302 | 316 |
| Evaporative Loss (NOACK), wt. % | None | D 5800 | 1.4 | 0.8 | 0.3 |
| Vapor Pressure - Reid (RVP), psi | None | D 5191 | ≈0 | ≈0 | ≈0 |

TABLE 9

| | | Estimated | Melting | Cloud | | | |
|---|---|---|---|---|---|---|---|
| | | EN | Pt. | Pt. | Visc. @ | Visc. @ | Visc. |
| Example # | Alcohol | (approx.) | ° C. | ° C. | 40° C. | 100° C. | Index |
| 8 | Jarcol ™ I-18CG | 2.0-2.6 | −15 | −13 | 103.4 | 16.6 | 174 |
| 8 | Jarcol ™ I-12 | 2.0-2.6 | −39 | −40 | 110.9 | 16.9 | 166 |
| 8 | Jarcol ™ I-20 | 2.0-2.6 | −42 | <−42 | 125.2 | 18.5 | 166 |
| 8 | Jarcol ™ I-16 | 2.0-2.6 | −51 | <−51 | 79.7 | 13.2 | 168 |
| 8 | Jarcol ™ 85BJ | 2.0-2.6 | −15 | −6 | 123.8 | 19.5 | 179 |
| 8 | Fineoxocol ® | 2.0-2.6 | −39 | −41 | 174.2 | 21.1 | 143 |
| | 180 | | | | | | |
| 8 | Jarcol ™ I-18T | 2.0-2.6 | −42 | <−42 | 130.8 | 19.2 | 167 |
| 8 | Isobutanol | 2.0-2.6 | −36 | −36 | 74.1 | 12.6 | 170 |
| 9 | Isobutanol | 1.5-2.2 | −36 | −36 | 59.5 | 10.6 | 170 |

### Example 11

Saturated and unsaturated estolides having varying acid values were subjected to several corrosion and deposit tests. These tests included the High Temperature Corrosion Bench Test (HTCBT) for several metals, the ASTM D130 corrosion test, and the MHT-4 TEOST (ASTM D7097) test for correlating piston deposits. The estolides tested having higher acid values (0.67 mg KOH/g) were produced using the method set forth in Examples 1 and 4 for producing Ex. 1 and Ex. 4A (Ex.1* and Ex.4A* below). The estolides tested having lower acid values (0.08 mg KOH/g) were produced using the method set forth in Examples 1 and 4 for producing Ex. 1 and Ex. 4A except the crude free-acid estolide was worked up and purified prior to esterification with BF₃.OET₂ (0.15 equiv.; reacted with estolide and 2-EH in Dean Stark trap at 80° C. in vacuo (10 torr abs) for 12 hrs while continuously being agitated; crude reaction product washed 4× H₂O; excess 2-EH removed by heating washed reaction product to 140° C. in vacuo (10 torr abs) for 1 hr) (Ex.4A# below). Estolides having an IV of 0 were hydrogenated via 10 wt. % palladium embedded on carbon at 75° C. for 3 hours under a pressurized hydrogen atmosphere (200 psig) (Ex.4A*H and Ex.4A#H below) The corrosion and deposit tests were performed with a Dexos™ additive package. Results were compared against a mineral oil standard:

TABLE 10

| | | | | Ex. | Ex. | Ex. |
|---|---|---|---|---|---|---|
| | Stan- | Ex. 1* | Ex. 4A* | 4A*H | 4A# | 4A#H |
| | dard | Estolide | Estolide | Estolide | Estolide | Estolide |
| Acid Value | — | ~0.7 | 0.67 | 0.67 | 0.08 | 0.08 |
| (mg KOH/g) | | | | | | |
| Iodine Value | — | ~45 | 16 | 0 | 16 | 0 |
| (IV) | | | | | | |
| HTCBT Cu | 13 | 739 | 279 | 60 | 9.3 | 13.6 |
| HTCBT Pd | 177 | 11,639 | 1,115 | 804 | 493 | 243 |
| HTCBT Sn | 0 | 0 | 0 | 0 | 0 | 0 |
| ASTM D130 | 1A | 4B | 3A | 1B | 1A | 1A |
| MHT-4 | 18 | 61 | 70 | 48 | 12 | 9.3 |

### Example 12

“Ready” and “ultimate” biodegradability of the estolide produced in Ex. 1 was tested according to standard OECD procedures. Results of the OECD biodegradability studies are set forth below in Table 11:

TABLE 11

| | 301D 28-Day | 302D Assay |
|---|---|---|
| | (% degraded) | (% degraded) |
| Canola Oil | 86.9 | 78.9 |
| Ex. 1 | 64.0 | 70.9 |
| Base Stock | | |

### Example 13

The Ex. 1 estolide base stock from Example 1 was tested under OECD 203 for Acute Aquatic Toxicity. The tests showed that the estolides are nontoxic, as no deaths were reported for concentration ranges of 5,000 mg/L and 50,000 mg/L.

### Example 14

Liquid-type lotion products were prepared by mixing together the following components: oil-phase component (10 wt. %); water (85 wt. %); 1,2-octanediol (0.25 wt. %); 1,2-hexanediol (0.25 wt. %); phenoxyethanol (1 wt. %); Polysorbate 20 (1 wt. %); Carbomer (0.5 wt. %); acrylates/C10—C30 alkyl acrylate crosspolymer (1 wt. %); and 10% aqueous sodium hydroxide solution (1 wt. %). Four sample lotion products were prepared, each with a different oil-phase component: Ex. 4A estolide (Ex. 14A lotion), isopropyl palmitate (Ex. 14B lotion), polydimethylsiloxane (Ex. 14C lotion), and sunflower seed oil (Ex. 14D lotion). A control lotion was also prepared without an oil-phase component, which was replaced with an additional 10 wt. % of water. The properties of those lotion products are compared below in Examples 15.

### Example 15

### Skincare Testing

Skin Barrier Function (Moisture Retention)—transepidermal water loss (TEWL) was tested on Ex. 14A-D lotions using a Delfin VapoMeter to determine the increase in relative humidity, which is used to calculate the evaporation rate value (g/m²h). Ex. 14A-D lotions were each applied to the volar forearm area of ten (10) test subjects. Baseline mean TEWL values for each area of application were determined at 0 hrs for each test subject. The change from baseline was then detected for each area of application at 2 hr, 4 hr, and 8 hr intervals. A positive change (increase) from the baseline represents an increase in TEWL. The mean value and change in TEWL from the baseline for all subjects and time intervals for each of the Ex. 14A-D lotions is reported below in Table 12.

TABLE 12

| | | 2 hrs | 4 hrs | 8 hrs |
|---|---|---|---|---|
| Formulation | Baseline | (change) | (change) | (change) |
| Control | 5.0 | 4.9 | 6.2 | 6.1 |
| | | (−0.1) | (+1.2) | (+1.1) |
| Ex. 14A | 7.4 | 5.6 | 6.0 | 6.4 |
| | | (−1.7) | (−1.4) | (−1.0) |
| Ex. 14B | 5.8 | 5.7 | 6.4 | 6.3 |
| | | (−0.2) | (+0.5) | (+0.4) |
| Ex. 14C | 6.4 | 5.4 | 6.0 | 6.1 |
| | | (−1.0) | (−0.4) | (−0.3) |
| Ex. 14D | 7.3 | 6.5 | 6.2 | 7.0 |
| | | (−0.8) | (−1.1) | (−0.3) |

Hydration/Moisturization—analytical tests were conducted on Ex. 14A-D lotions by using a Corneometer 825 PC®, which measured the dielectric constant occurring in the stratum corneum (upper most layer of skin). Ex. 14A-D lotions were each applied to the stratum corneum of the volar forearm of ten (10) test subjects. Baseline mean corneometer values (dielectric constants) for each area of application were determined at 0 hrs for each test subject. The change from baseline was then detected for each area of application at 2 hr, 4 hr, and 8 hr intervals. A positive change (increase) from the baseline represents an increased conductance and capacitance, wherein a higher capacitance represents a higher level of hydration in the stratum corneum. The mean value and change in capacitance hydration units from the baseline for all subjects and time intervals for each of the Ex. 14A-D lotions is reported below in Table 13.

TABLE 13

| | | 2 hrs | 4 hrs | 8 hrs |
|---|---|---|---|---|
| Formulation | Baseline | (change) | (change) | (change) |
| Control | 27.6 | 26.3 | 28.7 | 27.9 |
| | | (−1.3) | (+1.1) | (+2.3) |
| Ex. 14A | 25.1 | 28.5 | 29.7 | 29.3 |
| | | (+3.4) | (+4.6) | (+4.2) |
| Ex. 14B | 26.2 | 28.5 | 28.6 | 29.7 |
| | | (+2.3) | (+2.4) | (+3.5) |
| Ex. 14C | 26.5 | 26.1 | 26.2 | 26.2 |
| | | (−0.4) | (−0.3) | (−0.3) |
| Ex. 14D | 25.9 | 30.4 | 30.1 | 29.4 |
| | | (+4.5) | (+4.2) | (+3.5) |

### Safety Testing

Human Repeat Insult Patch Test (HRIPT)—patches containing Ex. 4A estolide were placed on the arms of fifty-one (51) subjects 3× a week for three weeks, 24 hrs at a time, for a total of nine (9) applications. The test was done to determine whether the estolide compound tested was essentially non-irritating and non-sensitizing. Evaluation criteria were subject to a scale of 0 to 4, wherein: 0=no visible skin reaction; 0.5=barely perceptible; 1=mild; 2=moderate; 3=marked; and 4=severe. Under supervised removal, all 51 subjects were reported to have no visible skin reaction (0 value) for each of the 9 applications.

1. A cosmetic formulation comprising
water;
at least one humectant;
at least one thickener;
at least one surfactant; and
at least one estolide compound selected from compounds of Formula I: 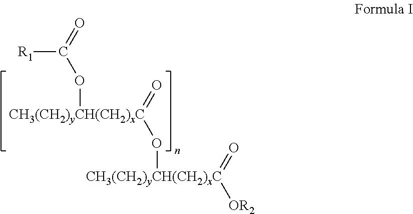
wherein
x is, independently for each occurrence, an integer selected from 0 to 20;
y is, independently for each occurrence, an integer selected from 0 to 20;
n is an integer greater than or equal to 0;
R₁ is an optionally substituted alkyl that is saturated or unsaturated, and branched or unbranched; and
R₂ is an optionally substituted unsubstituted alkyl that is saturated or unsaturated, and branched or unbranched,
wherein each fatty acid chain residue of said at least one estolide compound is independently optionally substituted.

2. The cosmetic formulation according to claim 1, wherein
x is, independently for each occurrence, an integer selected from 1 to 10;
y is, independently for each occurrence, an integer selected from 1 to 10;
n is an integer selected from 0 to 8;
R₁ is an optionally substituted C₁ to C₂₂ alkyl that is saturated or unsaturated, and branched or unbranched; and
R₂ is an optionally substituted C₁ to C₂₂ alkyl that is saturated or unsaturated, and branched or unbranched,
wherein each fatty acid chain residue is unsubstituted.

3. The cosmetic formulation according to claim 2, wherein
x+y is, independently for each chain, an integer selected from 13 to 15; and
n is an integer selected from 0 to 6.

4. (canceled)

5. The cosmetic formulation according to claim 1, wherein R₂ is a branched or unbranched C₁ to C₂₀ alkyl that is saturated or unsaturated.

6. The cosmetic formulation according to claim 5, wherein R₂ is selected from methyl, ethyl, propyl, butyl, pentyl, hexyl, heptyl, octyl, nonyl, decanyl, undecanyl, dodecanyl, tridecanyl, tetradecanyl, pentadecanyl, hexadecanyl, heptadecanyl, octadecanyl, nonadecanyl, and icosanyl, which are saturated or unsaturated and branched or unbranched.

7. The cosmetic formulation according to claim 5, wherein R₂ is selected from C₆ to C₁₂ alkyl.

8. The cosmetic formulation according to claim 7, wherein R₂ is 2-ethylhexyl.

9. The cosmetic formulation according to claim 1, wherein R₁ is a branched or unbranched C₁ to C₂₀ alkyl that is saturated or unsaturated.

10. The cosmetic formulation according to claim 9, wherein R₁ is selected from methyl, ethyl, propyl, butyl, pentyl, hexyl, heptyl, octyl, nonyl, decanyl, undecanyl, dodecanyl, tridecanyl, tetradecanyl, pentadecanyl, hexadecanyl, heptadecanyl, octadecanyl, nonadecanyl, and icosanyl, which are saturated or unsaturated and branched or unbranched.

11-52. (canceled)

53. The cosmetic formulation according to claim 1, wherein cosmetic formulation has an EN selected from an integer or fraction of an integer that is equal to or less than 2, wherein EN is the average number of linkages in compounds according to Formula I.

54-55. (canceled)

56. The cosmetic formulation according to claim 53, wherein said estolide base oil has a kinematic viscosity equal to or less than 55 cSt when measured at 40° C.

57-58. (canceled)

59. The cosmetic formulation according to claim 56, wherein said estolide base oil has a melting point equal to or lower than −25° C.

60-76. (canceled)

77. The cosmetic formulation according to claim 3, wherein x is, independently for each occurrence, an integer selected from 7 and 8.

78. The cosmetic formulation according to claim 3, wherein y is, independently for each occurrence, an integer selected from 7 and 8.

79. (canceled)

80. The cosmetic formulation according to claim 1, wherein said cosmetic formulation is a moisturizing formulation.

81-82. (canceled)

83. The cosmetic formulation according to claim 1, wherein the at least one humectant is selected from a polyol and a polyethylene glycol.

84. The cosmetic formulation according to claim 83, wherein the polyol is a C₂-C₆ polyol.

85. The cosmetic formulation according to claim 1, further comprising at least one preservative.

86. (canceled)

87. The cosmetic formulation according to claim 1, wherein the at least one surfactant is a non-ionic surfactant.

88. The cosmetic formulation according to claim 1, wherein the at least one surfactant is a polyoxyethylene sorbitol fatty acid ester.

89. (canceled)

90. The cosmetic formulation according to claim 1, wherein the at least one thickener is a polymer of acrylic acid.

91. The cosmetic formulation according to claim 1, further comprising at least one neutralizing agent.

92. The cosmetic formulation according to claim 91, wherein the at least one neutralizing agent is sodium hydroxide.

93. The cosmetic formulation according to claim 1, wherein the at least one estolide compound has an acid value of equal to or less than 0.5 mg KOH/g.

94. The cosmetic formulation according to claim 93, wherein the at least one estolide compound has an acid value of equal to or less than 0.1 mg KOH/g.
